(19) United States
(12) Patent Application Publication
(10) Pub. No.: US 2004/0170994 A1
(43) Pub. Date: Sep. 2, 2004

(54) DNA sequences for human tumour suppressor genes

(76) Inventors: David Frederick Callen, Malvern South Australia (AU); Gabriel Kremmidiotis, Flagstaff Hill (AU); Scott Whitmore, Golden Grove (AU); Alison Gardner, Brighton (AU); Jason Powell, Cumberland Park (AU)

Correspondence Address: JENKINS & WILSON, PA, 3100 TOWER BLVD, SUITE 1400, DURHAM, NC, 27707, US

(21) Appl. No.: 10/467,506

(22) PCT Filed: Feb. 12, 2002
(86) PCT No.: PCT/AU02/00138

(30) Foreign Application Priority Data
Feb. 12, 2001 (AU) PR 3052
Feb. 12, 2001 (AU) PR 3053
Feb. 12, 2001 (AU) PR 3054

(51) Int. Cl. C12Q 1/68; G01N 33/574

(52) U.S. Cl. 435/6; 435/7.23

(57) ABSTRACT

A method for the diagnosis of a predisposition thereto, in a patient, comprising the steps of: (1) establishing the level of expression of a gene selected from the group consisting of PR domain containing 7 protein (PRDM7), CDT1 DNA replication factor (CDT1), charged multivesicular body protein 1/chromoatin modifying protein 1 (CHMP1), BTG3 associated nucleoprotein (BANP), BNO224, BNO36, BNO34, BNO208, BNO230 and BNO44; and (2) comparing expression of the gene to a baseline established from expression in normal tissue controls; wherein substantial variance from the baseline indicates that the patient is susceptible to cancer.

### TECHNICAL FIELD

The present invention is concerned with DNA sequences from the 16q24.3 region which encode functional domains indicative of a potential role in the tumourigenic process.

### BACKGROUND ART

The development of human carcinomas has been shown to arise from the accumulation of genetic changes involving both positive regulators of cell function (oncogenes) and negative regulators (tumour suppressor genes). For a normal somatic cell to evolve into a metastatic tumour it requires changes at the cellular level, such as immortalisation, loss of contact inhibition and invasive growth capacity, and changes at the tissue level, such as evasion of host immune responses and growth restraints imposed by surrounding cells, and the formation of a blood supply for the growing tumour.

Molecular genetic studies of colorectal carcinoma have provided substantial evidence that the generation of malignancy requires the sequential accumulation of a number of genetic changes within the same epithelial stem cell of the colon. For a normal colonic epithelial cell to become a benign adenoma, progress to intermediate and late adenomas, and finally become a malignant cell, inactivating mutations in tumour suppressor genes and activating mutations in proto-oncogenes are required (Fearon and Vogelstein, 1990).

The employment of a number of techniques, such as loss of heterozygosity (LOH), comparative genomic hybridisation (CGH) and cytogenetic studies of cancerous tissue, all of which exploit chromosomal abnormalities associated with the affected cell, has aided in the identification of a number of tumour suppressor genes and oncogenes associated with a range of tumour types.

In one aspect, studies of cancers such as retinoblastoma and colon carcinoma have supported the model that LOH is a specific event in the pathogenesis of cancer and has provided a mechanism in which to identify the cancer causing genes. For instance in colorectal carcinoma, inherited forms of the disease have been mapped to the long arm of chromosome 5 while LOH at 5q has been reported in both the familial and sporadic versions of the disease. The APC tumour suppressor gene, mapping to this region, was subsequently shown to be involved (Groden et al., 1991). The model is further highlighted in Von Hippel-Lindau (VHL) syndrome, a rare disorder that predisposes individuals to a variety of tumours including clear cell carcinomas of the kidneys and islet cell tumours of the pancreas. Both sporadic and inherited cases of the syndrome show LOH for the short arm of chromosome 3 and somatic translocations involving 3p in sporadic tumours, and genetic linkage to the same region in affected families has also been observed. The VHL tumour suppressor gene has since been identified from this region of chromosome 3 and mutations in it have been detected in 100% of patients who carry a clinical diagnosis of VHL disease. In addition, the VHL gene is inactivated in approximately 50-80% of the more common sporadic form of renal clear cell carcinoma.

The genetic determinants involved in breast cancer are not as well defined as that of colon cancer due in part to the histological stages of breast cancer development being less well characterised. However, as with colon carcinoma, it is believed that a number of genes need to become involved in a stepwise progression during breast tumourigenesis.

Certain women appear to be at an increased risk of developing breast cancer. Genetic linkage analysis has shown that 5 to 10% of all breast cancers are due to at least two autosomal dominant susceptibility genes. Generally, women carrying a mutation in a susceptibility gene develop breast cancer at a younger age compared to the general population, often have bilateral breast tumours, and are at an increased risk of developing cancers in other organs, particularly carcinoma of the ovary.

Genetic linkage analysis on families showing a high incidence of early-onset breast cancer (before the age of 46) was successful in mapping the first susceptibility gene, BRCA1, to chromosome 17q21 (Hall et al., 1990). Subsequent to this, the BRCA2 gene was mapped to chromosome 13q12-q13 (Wooster et al., 1994) with this gene conferring a higher incidence of male breast cancer and a lower incidence of ovarian cancer when compared to BRCA1.

Both BRCA1 and BRCA2 have since been cloned (Miki et al., 1994; Wooster et al., 1995) and numerous mutations have been identified in these genes in susceptible individuals with familial cases of breast cancer.

Additional inherited breast cancer syndromes exist, however they are rare. Inherited mutations in the TP53 gene have been identified in individuals with Li-Fraumeni syndrome, a familial cancer resulting in epithelial neoplasms occurring at multiple sites including the breast. Similarly, germline mutations in the MMAC1/PTEN gene involved in Cowden's disease and the ataxia telangiectasia (AT) gene have been shown to confer an increased risk of developing breast cancer, among other clinical manifestations, but together account for only a small percentage of families with an inherited predisposition to breast cancer.

Somatic mutations in the TP53 gene have been shown to occur in a high percentage of individuals with sporadic breast cancer. However, although LOH has been observed at the BRCA1 and BRCA2 loci at a frequency of 30 to 40% in sporadic cases (Cleton-Jansen et al., 1995; Saito et al., 1993), there is virtually no sign of somatic mutations in the retained allele of these two genes in sporadic cancers (Futreal et al., 1994; Miki et al., 1996). Recent data suggests that DNA methylation of the promoter sequence of these genes may be an important mechanism of down-regulation. The use of both restriction fragment length polymorphisms and small tandem repeat polymorphic markers has identified numerous regions of allelic imbalance in breast cancer suggesting the presence of additional tumour suppressor genes, which may be implicated in breast cancer. Data compiled from more than 30 studies reveals the loss of DNA from at least 11 chromosome arms at a frequency of more than 25%, with regions such as 16q and 17p affected in more than 50% of tumours (Devilee and Cornelisse, 1994; Brenner and Aldaz, 1995). However only some of these regions are known to harbour tumour suppressor genes shown to be mutated in individuals with both sporadic (TP53 and RB genes) and familial (TP53, RB, BRCA1, and BRCA2 genes) forms of breast cancer.

Cytogenetic studies have implicated loss of the long arm of chromosome 16 as an early event in breast carcinogenesis since it is found in tumours with few or no other cytogenetic abnormalities. Alterations in chromosome 1 and 16 have also been seen in several cases of ductal carcinoma in situ (DCIS), the preinvasive stage of ductal breast carcinoma. In addition, LOH studies on DCIS samples identified loss of 16q markers in 29 to 89% of the cases tested (Chen et al., 1996; Radford et al., 1995). In addition, examination of tumours from other tissue types have indicated that 16q LOH is also frequently seen in prostate, lung, hepatocellular, ovarian, primitive neuroectodermal and Wilms' tumours.

Together, these findings suggest the presence of a tumour suppressor gene mapping to the long arm of chromosome 16 that is critically involved in the early development of a large proportion of breast cancers as well as cancers from other tissue types, but to date no such gene has been identified.

### DISCLOSURE OF THE INVENTION

The present invention provides nucleic acid and protein sequences that represent tumour suppressor genes involved in breast cancer, herein termed “tumour suppressor sequences”. The tumour suppressor sequences of the invention have been identified from a region of LOH seen in breast cancer, as well as other carcinomas including prostate tumours. Combined with the knowledge that these tumour suppressor sequences confer functional properties potentially linked with cancer to the proteins with which they encode suggests they are tumour suppressor genes playing a contributory role in cancer. The tumour suppressor sequences of the invention are described in Table 1 and are represented by SEQ ID NO:1 to 26.

The present invention also encompasses isolated nucleic acid and/or amino acid sequences which are homologous to the tumour suppressor sequences described above. Such homology is based on the overall nucleic acid or amino acid sequence of the group described in Table 1 and represented by the SEQ ID NO:1 to 26 and is determined using either homology programs or hybridisation conditions as outlined below.

A nucleic acid or protein is a tumour suppressor nucleic acid or protein if the overall homology of the nucleic acid or protein sequence to one of the sequences described in Table 1 and represented by the SEQ ID NO:1 to 26 is at least 70%, preferably 85% and most preferably 95%. Homology in this context means sequence similarity or identity, with identity being preferred.

In a preferred embodiment, the sequences which are used to determine sequence identity or similarity are selected from the sequences described in Table 1 and represented by the SEQ ID NO:1 to 26 or are naturally occurring allelic variants, sequence variants or splice variants of these sequences.

Sequence identity is typically calculated using the BLAST algorithm, described in Altschul et al Nucleic Acids Res. 25, 3389-3402 (1997) with the BLOSUM62 default matrix.

In one embodiment, nucleic acid homology can be determined through hybridisation studies. Nucleic acids which hybridise under stringent conditions to the nucleic acids of the invention are considered breast cancer sequences. Under stringent conditions, hybridisation will most preferably occur at 42° C. in 750 mM NaCl, 75 mM trisodium citrate, 2% SDS, 50% formamide, 1× Denhart's, 10% (w/v) dextran sulphate and 100 μg/ml denatured salmon sperm DNA. Useful variations on these conditions will be readily apparent to those skilled in the art. The washing steps which follow hybridization most preferably occur at 65° C. in 15 mM NaCl, 1.5 mM trisodium citrate, and 1% SDS. Additional variations on these conditions will be readily apparent to those skilled in the art.

In a further aspect, the invention provides tumour suppressor sequences, as described in Table 1 and represented by the SEQ ID NO:1 to 26, or the nucleotide sequence of a nucleic acid which hybridises thereto as described above, and appropriate control elements of the tumour suppressor sequences.

Preferably the control elements are those which mediate expression in breast tissue, but may also mediate expression in other tissues including, but not restricted to, prostate, liver and ovary. The tumour suppressor nucleic acid sequences of the present invention can be engineered using methods accepted in the art so as to alter the sequences for a variety of purposes. These include, but are not limited to, modification of the cloning, processing, and/or expression of the gene product. PCR reassembly of gene fragments and the use of synthetic oligonucleotides allow the engineering of breast cancer sequences of the invention. For example, oligonucleotide-mediated site-directed mutagenesis can introduce mutations that create new restriction sites, alter glycosylation patterns and produce splice variants etc.

As a result of the degeneracy of the genetic code, a number of polynucleotide sequences encoding tumour suppressor proteins of the invention, some that may have minimal similarity to the polynucleotide sequences of any known and naturally occurring gene, may be produced. Thus, the invention includes each and every possible variation of polynucleotide sequence that could be made by selecting combinations based on possible codon choices. These combinations are made in accordance with the standard triplet genetic code as applied to the polynucleotide sequence of naturally occurring breast cancer sequences, and all such variations are to be considered as being specifically disclosed.

The polynucleotides of this invention include RNA, cDNA, genomic DNA, synthetic forms, and mixed polymers, both sense and antisense strands, and may be chemically or biochemically modified, or may contain non-natural or derivatised nucleotide bases as will be appreciated by those skilled in the art. Such modifications include labels, methylation, intercalators, alkylators and modified linkages. In some instances it may be advantageous to produce nucleotide sequences encoding tumour suppressor sequences of the invention, or their derivatives, possessing a substantially different codon usage than that of the naturally occurring gene. For example, codons may be selected to increase the rate of expression of the peptide in a particular prokaryotic or eukaryotic host corresponding with the frequency that particular codons are utilized by the host. Other reasons to alter the nucleotide sequence encoding tumour suppressor sequences of the invention, or their derivatives, without altering the encoded amino acid sequences include the production of RNA transcripts having more desirable properties, such as a greater half-life, than transcripts produced from the naturally occurring sequence.

The invention also encompasses production of tumour suppressor sequences of the invention entirely by synthetic chemistry. Synthetic sequences may be inserted into expression vectors and cell systems that contain the necessary elements for transcriptional and translational control of the inserted coding sequence in a suitable host. Numerous types of appropriate expression vectors and suitable regulatory elements are known in the art for a variety of host cells. Regulatory elements may include regulatory, sequences, promoter sequences, ribosomal binding sites, transcriptional start and stop sequences, 5′ and 3′ untranslated regions and specific translational start and stop signals (such as an ATG initiation codon and Kozak consensus sequence). Regulatory elements will allow more efficient translation of sequences encoding breast cancer genes of the invention. In cases where the complete tumour suppressor coding sequence including its initiation codon and upstream regulatory sequences are inserted into the appropriate expression vector, additional control signals may not be needed. However, in cases where only coding sequence, or a fragment thereof, is inserted, exogenous translational control signals as described above should be provided by the vector. Such signals may be of various origins, both natural and synthetic. The efficiency of expression may be enhanced by the inclusion of enhancers appropriate for the particular host cell system used (Scharf et al., 1994).

The present invention allows for the preparation of purified tumour suppressor polypeptide or protein, from the polynucleotides of the present invention or variants thereof. In order to do this, host cells may be transfected with a nucleic acid molecule as described above. Typically said host cells are transfected with an expression vector comprising a nucleic acid encoding a tumour suppressor protein according to the invention. Cells are cultured under the appropriate conditions to induce or cause expression of the tumour suppressor protein. The conditions appropriate for protein expression will vary with the choice of the expression vector and the host cell, and will be easily ascertained by one skilled in the art.

A variety of expression vector/host systems may be utilized to contain and express the tumour suppressor sequences of the invention and are well known in the art. These include, but are not limited to, microorganisms such as bacteria transformed with plasmid or cosmid DNA expression vectors; yeast transformed with yeast expression vectors; insect cell systems infected with viral expression vectors (e.g., baculovirus); or mouse or other animal or human tissue cell systems. In a preferred embodiment the tumour suppressor proteins of the invention are expressed in mammalian cells using various expression vectors including plasmid, cosmid and viral systems such as adenoviral, retroviral or vaccinia virus expression systems. The invention is not limited by the host cell employed.

The polynucleotide sequences, or variants thereof, of the present invention can be stably expressed in cell lines to allow long-term production of recombinant proteins in mammalian systems. These sequences can be transformed into cell lines using expression vectors which may contain viral origins of replication and/or endogenous expression elements and a selectable marker gene on the same or on a separate vector. The selectable marker confers resistance to a selective agent, and its presence allows growth and recovery of cells which successfully express the introduced sequences. Resistant clones of stably transformed cells may be propagated using tissue culture techniques appropriate to the cell type.

The protein produced by a transformed cell may be secreted or retained intracellularly depending on the sequence and/or the vector used. As will be understood by those of skill in the art, expression vectors containing polynucleotides which encode a protein of the invention may be designed to contain signal sequences which direct secretion of the protein through a prokaryotic or eukaryotic cell membrane.

In addition, a host cell strain may be chosen for its ability to modulate expression of the inserted sequences or to process the expressed protein in the desired fashion. Such modifications of the polypeptide include, but are not limited to, acetylation, glycosylation, phosphorylation, and acylation. Post-translational cleavage of a “prepro” form of the protein may also be used to specify protein targeting, folding, and/or activity. Different host cells having specific cellular machinery and characteristic mechanisms for post-translational activities (e.g., CHO or HeLa cells), are available from the American Type Culture Collection (ATCC) and may be chosen to ensure the correct modification and processing of the foreign protein.

When large quantities of protein are needed such as for antibody production, vectors which direct high levels of expression of the breast cancer sequences may be used such as those containing the T5 or T7 inducible bacteriophage promoter. The present invention also includes the use of the expression systems described above in generating and isolating fusion proteins which contain the important functional domains of the protein. These fusion proteins are used for binding, structural and functional studies as well as for the generation of appropriate antibodies.

In order to express and purify the protein as a fusion protein, the appropriate cDNA sequence is inserted into a vector which contains a nucleotide sequence encoding another peptide (for example, glutathionine succinyl transferase). The fusion protein is expressed and recovered from prokaryotic or eukaryotic cells. The fusion protein can then be purified by affinity chromatography based upon the fusion vector sequence. The relevant protein can subsequently be obtained by enzymatic cleavage of the fusion protein.

In one embodiment, a fusion protein may be generated by the fusion of a tumour suppressor polypeptide with a tag polypeptide which provides an epitope to which an anti-tag antibody can selectively bind. The epitope tag is generally placed at the amino- or carboxy-terminus of the tumour suppressor polypeptide. The presence of such epitope-tagged forms of a tumour suppressor polypeptide can be detected using an antibody against the tag polypeptide. Also, provision of the epitope tag enables the tumour suppressor polypeptide to be readily purified by affinity purification using an anti-tag antibody or another type of affinity matrix that binds to the epitope tag.

Various tag polypeptides and their respective antibodies are well known in the art. Examples include poly-histidine or poly-histidine-glycine tags and the c-myc tag and antibodies thereto.

Fragments of tumour suppressor polypeptide may also be produced by direct peptide synthesis using solid-phase; techniques. Automated synthesis may be achieved by using the ABI 431A Peptide Synthesizer (Perkin-Elmer). Various fragments of breast cancer polypeptide may be synthesized separately and then combined to produce the full-length molecule.

In a further aspect of the invention there is provided a method of preparing a polypeptide as described above, comprising the steps of:

(1) culturing the host cells under conditions effective for production of the polypeptide; and

(2) harvesting the polypeptide.

Substantially purified tumour suppressor proteins or fragments thereof can then be used in further biochemical analyses to establish secondary and tertiary structure for example by x-ray crystallography of the protein or by nuclear magnetic resonance (NMR). Determination of structure allows for the rational design of pharmaceuticals to interact with the protein, alter protein charge configuration or charge interaction with other proteins, or to alter its function in the cell.

According to the invention there is provided a method for the diagnosis of a predisposition to cancer, in a patient, comprising the steps of:

(1) establishing the level of expression of a gene selected from the group consisting of PR domain containing 7 protein (PRDM7), CDT1 DNA replication factor (CDT1), charged multivesicular body protein 1/chromoatin modifying protein 1 (CHMP1), BTG3 associated nucleoprotein (BANP), BNO224, BNO36, BNO34, BNO208, BNO230 and BNO44; and

(2) comparing expression of the gene to a baseline established from expression in normal tissue controls;

wherein substantial variance from the baseline indicates that the patient is susceptible to cancer.

The tumour suppressor sequences of the present invention have been identified from a region of restricted LOH seen in breast cancer. In addition, these tumour suppressor sequences have been shown to confer functional properties potentially linked with cancer to the proteins with which they encode. As many of these genes are expressed in a wide variety of tissues and LOH of 16q has been found in cancers of other tissue types, including prostate, liver, ovary, primitive neuroectodermal and Wilms' tumours, suggests they may represent tumour suppressor genes involved in a variety of cancers. Such cancers may include, but are not limited to adenocarcinoma, leukemia, lymphoma, melanoma, myeloma, sarcoma, teratocarcinoma, and, in particular, cancers of the breast, prostate, blood, germ cells, liver, ovary, adrenal gland, cervix, heart, brain, lung, placenta, skeletal muscle, synovial membrane, tonsil, lymph tissue, kidney, colon, uterus, skin and testis. Other cancers may include those of the head and neck, bladder, bone, bone marrow, gall bladder, ganglia, gastrointestinal tract, pancreas, parathyroid, penis, salivary glands, spleen, stomach, thymus and thyroid gland.

With the identification of the tumour suppressor nucleotide and protein sequences of the invention, probes and antibodies raised to the genes can be used in a variety of hybridisation and immunological assays to screen for and detect the presence of either a normal or mutated gene or gene product.

The nucleotide and protein sequences of the tumour suppressor genes provided in this invention also enable therapeutic methods for the treatment of cancers associated with one or more of these genes, and enable methods for the diagnosis or prognosis of all cancers associated with the these genes. Examples of such cancers include, but are not limited to, those listed above.

Due to their recessive nature, both copies of a tumour suppressor gene within a cell need to be inactivated for that cell to be affected. Therefore, in the treatment of cancers associated with inactivated or decreased tumour suppressor gene activity and/or expression, it is desirable to increase the activity and/or expression of the relevant gene.

Enhancing Tumour Suppressor Gene or Protein Function

Enhancing, stimulating or re-activating the function of those tumour suppressor genes or proteins that are mutated or down-regulated in cancer can be achieved in a variety of ways as would be appreciated by those skilled in the art.

In a preferred embodiment a tumour suppressor gene of the invention is administered to a subject to treat or prevent a cancer associated with decreased activity and/or expression of the gene.

In a further aspect, there is provided the use of a nucleic acid molecule of the invention, as described above, in the manufacture of a medicament for the treatment of a cancer associated with decreased activity and/or expression of the corresponding gene.

Typically, a vector capable of expressing a tumour suppressor gene of the invention, or fragment or derivative thereof, may be administered to a subject to treat or prevent a cancer associated with decreased activity and/or expression of the gene, including but not limited to, those described above.

Transducing retroviral vectors are often used for somatic cell gene therapy because of their high efficiency of infection and stable integration and expression. The full-length breast cancer gene, or portions thereof, can be cloned into a retroviral vector and expression can be driven from its endogenous promoter or from the retroviral long terminal repeat or from a promoter specific for the target cell type of interest. Other viral vectors can be used and include, as is known in the art, adenoviruses, adeno-associated virus, vaccinia virus, papovaviruses, lentiviruses and retroviruses of avian, murine and human origin.

Gene therapy would be carried out according to established methods (Friedman, 1991; Culver, 1996). A vector containing a copy of a tumour suppressor gene linked to expression control elements and capable of replicating inside the cells is prepared. Alternatively the vector may be replication deficient and may require helper cells or helper virus for replication and virus production and use in gene therapy.

Gene transfer using non-viral methods of infection can also be used. These methods include direct injection of DNA, uptake of naked DNA in the presence of calcium phosphate, electroporation, protoplast fusion or liposome delivery. Gene transfer can also be achieved by delivery as a part of a human artificial chromosome or receptor-mediated gene transfer. This involves linking the DNA to a targeting molecule that will bind to specific cell-surface receptors to induce endocytosis and transfer of the DNA into mammalian cells. One such technique uses poly-L-lysine to link asialoglycoprotein to DNA. An adenovirus is also added to the complex to disrupt the lysosomes and thus allow the DNA to avoid degradation and move to the nucleus. Infusion of these particles intravenously has resulted in gene transfer into hepatocytes.

In affected subjects that express a mutated form of a tumour suppressor gene of the invention, it may be possible to prevent the cancer by introducing into the affected cells a wild-type copy of the gene such that it recombines with the mutant gene. This requires a double recombination event for the correction of the gene mutation. Vectors for the introduction of genes in these ways are known in the art, and any suitable vector may be used. Alternatively, introducing another copy of the gene bearing a second mutation in that gene may be employed so as to negate the original gene mutation and block any negative effect.

In a still further aspect the invention provides a method for the treatment of a cancer associated with decreased activity and/or expression of a tumour suppressor gene of the invention, comprising administering a polypeptide as described above, or an agonist thereof, to a subject in need of such treatment.

In another aspect the invention provides the use of a polypeptide as described above, or an agonist thereof, in the manufacture of a medicament for the treatment of a cancer associated with decreased activity and/or expression of a tumour suppressor gene.

Diagnostic and Prognostic Applications

Polynucleotide sequences encoding the tumour suppressor genes of the invention may be used for the diagnosis or prognosis of cancers associated with their dysfunction, or a predisposition to such cancers. Examples of such cancers include, but are not limited to, adenocarcinoma, leukemia, lymphoma, melanoma, myeloma, sarcoma, teratocarcinoma, and, in particular, cancers of the breast, prostate, blood, germ cells, liver, ovary, adrenal gland, cervix, heart, brain, lung, placenta, skeletal muscle, synovial membrane, tonsil, lymph tissue, kidney, colon, uterus, skin and testis. Other cancers may include those of the head and neck, bladder, bone, bone marrow, gall bladder, ganglia, gastrointestinal tract, pancreas, parathyroid, penis, salivary glands, spleen, stomach, thymus and thyroid gland.

Diagnosis or prognosis may be used to determine the severity, type or stage of the disease state in order to initiate an appropriate therapeutic intervention.

In another embodiment of the invention, the polynucleotides that may be used for diagnostic or prognostic purposes include oligonucleotide sequences, genomic DNA and complementary RNA and DNA molecules. The polynucleotides may be used to detect and quantitate gene expression in biopsied tissues in which mutations or abnormal expression of the relevant tumour suppressor gene may be correlated with disease. Genomic DNA used for the diagnosis or prognosis may be obtained from body cells, such as those present in the blood, tissue biopsy, surgical specimen, or autopsy material. The DNA may be isolated and used directly for detection of a specific sequence or may be amplified by the polymerase chain reaction (PCR) prior to analysis. Similarly, RNA or cDNA may also be used, with or without PCR amplification. To detect a specific nucleic acid sequence, direct nucleotide sequencing, reverse transcriptase PCR (RT-PCR), hybridization using specific oligonucleotides, restriction enzyme digest and mapping, PCR mapping, RNAse protection, and various other methods may be employed. Oligonucleotides specific to particular sequences can be chemically synthesized and labelled radioactively or non-radioactively and hybridised to individual samples immobilized on membranes or other solid-supports or in solution. The presence or absence of a particular target tumour suppressor may then be visualized using methods such as autoradiography, fluorometry, or colorimetry.

In a particular aspect, the nucleotide sequences encoding a tumour suppressor gene of the invention may be useful in assays that detect the presence of associated disorders, particularly those mentioned previously. The nucleotide sequences encoding the relevant tumour suppressor gene may be labelled by standard methods and added to a fluid or tissue sample from a patient under conditions suitable for the formation of hybridization complexes. After a suitable incubation period, the sample is washed and the signal is quantitated and compared with a standard value. If the amount of signal in the patient sample is significantly altered in comparison to a control sample then the presence of altered levels of nucleotide sequences encoding the tumour suppressor gene in the sample indicates the presence of the associated disorder. Such assays may also be used to evaluate the efficacy of a particular therapeutic treatment regimen in animal studies, in clinical trials, or to monitor the treatment of an individual patient.

In order to provide a basis for the diagnosis or prognosis of a disorder associated with a mutation in a particular tumour suppressor gene of the invention, the nucleotide sequence of the relevant gene can be compared between normal tissue and diseased tissue in order to establish whether the patient expresses a mutant gene.

In order to provide a basis for the diagnosis or prognosis of a disorder associated with decreased expression of a particular tumour suppressor gene of the invention, a normal or standard profile for expression is established. This may be accomplished by combining body fluids or cell extracts taken from normal subjects, either animal or human, with a sequence, or a fragment thereof, encoding the relevant breast cancer gene, under conditions suitable for hybridization or amplification. Standard hybridization may be quantified by comparing the values obtained from normal subjects with values from an experiment in which a known amount of a substantially purified polynucleotide is used. Another method to identify a normal or standard profile for expression of a particular tumour suppressor gene is through quantitative RT-PCR studies. RNA isolated from body cells of a normal individual, particularly RNA isolated from tumour cells, is reverse transcribed and real-time PCR using oligonucleotides specific for the relevant tumour suppressor gene are conducted to establish a normal level of expression of the gene.

Standard values obtained in both these examples may be compared with values obtained from samples from patients who are symptomatic for a disorder. Deviation from standard values is used to establish the presence of a disorder.

Once the presence of a disorder is established and a treatment protocol is initiated, hybridization assays or quantitative RT-PCR studies may be repeated on a regular basis to determine if the level of expression in the patient begins to approximate that which is observed in the normal subject. The results obtained from successive assays may be used to show the efficacy of treatment over a period ranging from several days to months.

In one aspect, hybridization with PCR probes which are capable of detecting polynucleotide sequences, including genomic sequences, encoding a particular tumour suppressor gene, or closely related molecule, may be used to identify nucleic acid sequences which encode the gene. The specificity of the probe, whether it is made from a highly specific region, e.g., the 5′ regulatory region, or from a less specific region, e.g., a conserved motif, and the stringency of the hybridization or amplification will determine whether the probe identifies only naturally occurring sequences encoding the tumour suppressor gene, allelic variants, or related sequences.

Probes may also be used for the detection of related sequences, and should preferably have at least 50% sequence identity to any of the tumour suppressor encoding sequences. The hybridization probes of the subject invention may be DNA or RNA and may be derived from the sequence of SEQ ID Numbers: 1-26 or from genomic sequences including promoters, enhancers, and introns of the genes.

Means for producing specific hybridization probes for DNAs encoding the tumour suppressor genes of the invention include the cloning of polynucleotide sequences encoding these genes or their derivatives into vectors for the production of mRNA probes. Such vectors are known in the art, and are commercially available. Hybridization probes may be labelled by radionuclides such as ³²P or ³⁵S, or by enzymatic labels, such as alkaline phosphatase coupled to the probe via avidin/biotin coupling systems, or other methods known in the art.

According to a further aspect of the invention there is provided the use of a polypeptide as described above in the diagnosis or prognosis of a cancer associated with a tumour suppressor gene of the invention, or a predisposition to such cancers.

When a diagnostic or prognostic assay is to be based upon a tumour suppressor protein, a variety of approaches are possible. For example, diagnosis or prognosis can be achieved by monitoring differences in the electrophoretic mobility of normal and mutant proteins. Such an approach will be particularly useful in identifying mutants in which charge substitutions are present, or in which insertions, deletions or substitutions have resulted in a significant change in the electrophoretic migration of the resultant protein. Alternatively, diagnosis may be based upon differences in the proteolytic cleavage patterns of normal and mutant proteins, differences in molar ratios of the various amino acid residues, or by functional assays demonstrating altered function of the gene products.

In another aspect, antibodies that specifically bind a tumour suppressor protein of the invention may be used for the diagnosis or prognosis of cancers characterized by reduced activity and/or expression of the gene, or in assays to monitor patients being treated with the gene. Such antibodies may include, but are not limited to, polyclonal, monoclonal, chimeric and single chain antibodies as would be understood by the person skilled in the art.

For the production of antibodies, various hosts including rabbits, rats, goats, mice, humans, and others may be immunized by injection with a protein of the invention or with any fragment or oligopeptide thereof, which has immunogenic properties. Various adjuvants may be used to increase immunological response and include, but are not limited to, Freund's, mineral gels such as aluminum hydroxide, and surface-active substances such as lysolecithin. Adjuvants used in humans include BCG (bacilli Calmette-Guerin) and Corynebacterium parvum.

It is preferred that the oligopeptides, peptides, or fragments used to induce antibodies to the tumour suppressor proteins of the invention have an amino acid sequence consisting of at least about 5 amino acids, and, more preferably, of at least about 10 amino acids. It is also preferable that these oligopeptides, peptides, or fragments are identical to a portion of the amino acid sequence of the natural protein and contain the entire amino acid sequence of a small, naturally occurring molecule. Short stretches of amino acids from these proteins may be fused with those of another protein, such as KLH, and antibodies to the chimeric molecule may be produced.

Monoclonal antibodies to tumour suppressor proteins of the invention may be prepared using any technique which provides for the production of antibody molecules by continuous cell lines in culture. These include, but are not limited to, the hybridoma technique, the human B-cell hybridoma technique, and the EBV-hybridoma technique. (For example, see Kohler et al., 1975; Kozbor et al., 1985; Cote et al., 1983; Cole et al., 1984).

Antibodies may also be produced by inducing in vivo production in the lymphocyte population or by screening immunoglobulin libraries or panels of highly specific binding reagents as disclosed in the literature. (For example, see Orlandi et al., 1989; Winter et al., 1991).

Antibody fragments which contain specific binding sites for the tumour suppressor proteins may also be generated. For example, such fragments include, F(ab′)₂ fragments produced by pepsin digestion of the antibody molecule and Fab fragments generated by reducing the disulfide bridges of the F(ab′)₂ fragments. Alternatively, Fab expression libraries may be constructed to allow rapid and easy identification of monoclonal Fab fragments with the desired specificity. (For example, see Huse et al., 1989).

Various immunoassays may be used for screening to identify antibodies having the desired specificity. Numerous protocols for competitive binding or immunoradiometric assays using either polyclonal or monoclonal antibodies with established specificities are well known in the art. Such immunoassays typically involve the measurement of complex formation between a protein and its specific antibody. A two-site, monoclonal-based immunoassay utilizing monoclonal antibodies reactive to two non-interfering epitopes is preferred, but a competitive binding assay may also be employed.

Diagnostic or prognostic assays based on antibodies generated to the tumour suppressor proteins of the invention include methods that utilize the antibody and a label which will detect binding of the antibody to the appropriate protein in human body fluids or in extracts of cells or tissues. The antibodies may be used with or without modification, and may be labelled by covalent or non-covalent attachment of a reporter molecule.

A variety of protocols for measuring antibody binding include ELISAS, RIAS, and FACS, and are known in the art. These methods provide a basis for diagnosing altered or abnormal levels of tumour suppressor gene expression. Normal or standard values for tumour suppressor gene expression are established by combining body fluids or cell extracts taken from normal mammalian subjects, preferably human, with antibody to the appropriate tumour suppressor protein under conditions suitable for complex formation. The amount of standard complex formation may be quantitated by various methods, preferably by photometric means. Quantities of any of the tumour suppressor genes expressed in subject, control, and disease samples from biopsied tissues are compared with the standard values. Deviation between standard and subject values establishes the parameters for diagnosing disease.

Once an individual has been diagnosed with a cancer, effective treatments can be initiated. These may include administering a selective agonist to the relevant mutant tumour suppressor gene so as to restore its function to a normal level or introduction of the wild-type gene, particularly through gene therapy approaches as described above. Typically, a vector capable of expressing the appropriate full-length tumour suppressor gene or a fragment or derivative thereof may be administered. In an alternative approach to therapy, a substantially purified breast cancer polypeptide and a pharmaceutically acceptable carrier may be administered, as described above.

The nucleotide and protein sequences of the invention provide the ability to identify proteins that interact with these sequences. Interacting proteins may give an insight into the biological pathways in which the tumour suppressor proteins participate. In turn, proteins within these pathways may provide suitable targets for therapeutic applications.

The activity and/or expression of proteins within these pathways can subsequently be modulated in a number of ways so as to ultimately mimic the action that the wild-type tumour suppressor gene normally plays within the pathway. Methods include administering an antagonist of the proteins within the pathway to a subject in need of such treatment, such as an antibody designed to the protein or small molecule interactor, or through the use of antisense technologies such as antisense oligonucleotides, ribozymes, DNAzymes, injection of antisense RNA and transfection of antisense RNA expression vectors.

Drug Screening

The tumour suppressor genes of the invention may be used to construct eukaryotic cell lines which carry mutations in a particular gene of interest. The host cell lines are also defective at the polypeptide level. Other cell lines may be used where the expression of the tumour suppressor gene of interest can be switched off. The host cell lines or cells are grown in the presence of various drug compounds and the rate of growth of the host cells is measured to determine if the compound is capable of regulating the growth of defective cells.

Candidate pharmaceutical agents or compounds encompass numerous chemical classes, though typically they are organic molecules, preferably small organic compounds having molecular weight of more than 100 and less than about 2,500 daltons. Candidate agents are also found among biomolecules including peptides, saccharides, fatty acids and steroids. In this case peptides are preferred.

Compounds developed as a result of combinatorial library technology may also be screened. This provides a way to test a large number of different substances for their ability to restore wild-type function to the cells which contain a dysfunctional tumour suppressor gene. The use of peptide libraries is preferred (see patent WO97/02048) with such libraries and their use known in the art.

A substance identified as a modulator of cell growth and function may be peptide or non-peptide in nature. Non-peptide “small molecules” are often preferred for many in vivo pharmaceutical applications. In addition, a mimic or mimetic of the substance may be designed for pharmaceutical use. The design of mimetics based on a known pharmaceutically active compound (“lead” compound) is a common approach to the development of novel pharmaceuticals. This is often desirable where the original active compound is difficult or expensive to synthesise or where it provides an unsuitable method of administration. In the design of a mimetic, particular parts of the original active compound that are important in determining the target property are identified. These parts or residues constituting the active region of the compound are known as its pharmacophore. Once found, the pharmacophore structure is modelled according to its physical properties using data from a range of sources including x-ray diffraction data and NMR. A template molecule is then selected onto which chemical groups which mimic the pharmacophore can be added. The selection can be made such that the mimetic is easy to synthesise, is likely to be pharmacologically acceptable, does not degrade in vivo and retains the biological activity of the lead compound. Further optimisation or modification can be carried out to select one or more final mimetics useful for in vivo or clinical testing.

It is also possible to isolate a target-specific antibody and then solve its crystal structure. In principle, this approach yields a pharmacophore upon which subsequent drug design can be based as described above. It may be possible to avoid protein crystallography altogether by generating anti-idiotypic antibodies (anti-ids) to a functional, pharmacologically active antibody. As a mirror image of a mirror image, the binding site of the anti-ids would be expected to be an analogue of the original binding site. The anti-id could then be used to isolate peptides from chemically or biologically produced peptide banks.

Proteins involved in the tumour suppressor gene pathway may also be used for the screening of candidate pharmaceutical agents or compounds that interact with the proteins, for the treatment of cancers associated with the tumour suppressor dysfunction.

Agent screening techniques may include utilising eukaryotic or prokaryotic host cells that are stably transformed with recombinant molecules expressing a particular tumour suppressor pathway polypeptide preferably in competitive binding assays. Binding assays will measure for the formation of complexes between the polypeptide of interest and the agent being tested, or will measure the degree to which an agent being tested will interfere with the formation of a complex between the polypeptide of interest and a known ligand.

Another technique for drug screening provides high-throughput screening for compounds having suitable binding affinity to a tumour suppressor pathway polypeptide !of interest (see PCT published application WO84/03564). In this stated technique, large numbers of small peptide test compounds can be synthesised on a solid substrate and can be assayed through polypeptide binding and washing. Bound polypeptide is then detected by methods well known in the art. In a variation of this technique, purified polypeptides can be coated directly onto plates to identify interacting test compounds.

A tumour suppressor pathway polypeptide of interest may also be used for screening compounds developed as a result of combinatorial library technology as described above.

In a further aspect a pharmaceutical composition and a pharmaceutically acceptable carrier may be administered. The pharmaceutical composition may comprise any one or more of a polypeptide as described above, typically a substantially purified tumour suppressor polypeptide, an antibody to a tumour suppressor polypeptide, a vector capable of expressing a tumour suppressor polypeptide, a compound which increases expression of a tumour suppressor gene or a candidate drug that restores wild-type activity to a tumour suppressor gene.

The pharmaceutical composition may be administered to a subject to treat or prevent a cancer associated with decreased activity and/or expression of a tumour suppressor gene including, but not limited to, those provided above. Pharmaceutical compositions in accordance with the present invention are prepared by mixing a polypeptide of the invention, or active fragments or variants thereof, having the desired degree of purity, with acceptable carriers, excipients, or stabilizers, which are well known. Acceptable carriers, excipients or stabilizers are nontoxic at the dosages and concentrations employed, and include buffers such as phosphate, citrate, and other organic acids; antioxidants including absorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitrol or sorbitol; salt-forming counterions such as sodium; and/or nonionic surfactants such as Tween, Pluronics or polyethylene glycol (PEG).

Any of the therapeutic methods described above may be applied to any subject in need of such therapy, including, for example, mammals such as dogs, cats, cows, horses, rabbits, monkeys, and most preferably, humans.

Microarray

In further embodiments, complete cDNAs, oligonucleotides or longer fragments derived from any of the polynucleotide sequences described herein may be used as targets in a microarray. The microarray can be used to monitor the expression level of large numbers of genes simultaneously and to identify genetic variants, mutations, and polymorphisms. This information may be used to determine gene function, to understand the genetic basis of a disorder, to diagnose or prognose a disorder, and to develop and monitor the activities of therapeutic agents. Microarrays may be prepared, used, and analyzed using methods known in the art. (For example, see Schena et al., 1996; Heller et al., 1997).

Transformed Hosts

The present invention also provides for the production of genetically modified (knock-out, knock-in and transgenic), non-human animal models transformed with the DNA molecules of the invention. These animals are useful for the study of tumour suppressor gene function, to study the mechanisms of cancer as related to the tumour suppressor genes, for the screening of candidate pharmaceutical compounds, for the creation of explanted mammalian cell cultures which express the protein or mutant protein and for the evaluation of potential therapeutic interventions.

One of the tumour suppressor genes of the invention may have been inactivated by knock-out deletion, and knock-out genetically modified non-human animals are therefore provided.

Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, genetically modified mice and rats are highly desirable due to their relative ease of maintenance and shorter life spans. For certain studies, transgenic yeast or invertebrates may be suitable and preferred because they allow for rapid screening and provide for much easier handling. For longer-term studies, non-human primates may be desired due to their similarity with humans.

To create an animal model for a mutated tumour suppressor gene several methods can be employed. These include generation of a specific mutation in a homologous animal gene, insertion of a wild type human gene and/or a humanized animal gene by homologous recombination, insertion of a mutant (single or multiple) human gene as genomic or minigene cDNA constructs using wild type or mutant or artificial promoter elements or insertion of artificially modified fragments of the endogenous gene by homologous recombination. The modifications include insertion of mutant stop codons, the deletion of DNA sequences, or the inclusion of recombination elements (lox p sites) recognized by enzymes such as Cre recombinase.

To create a transgenic mouse, which is preferred, a mutant version of a particular breast cancer gene of the invention can be inserted into a mouse germ line using standard techniques of oocyte microinjection or transfection or microinjection into embryonic stem cells. Alternatively, if it is desired to inactivate or replace the endogenous breast cancer gene, homologous recombination using embryonic stem cells may be applied.

For oocyte injection, one or more copies of the mutant or wild type tumour suppressor gene can be inserted into the pronucleus of a just-fertilized mouse oocyte. This oocyte is then reimplanted into a pseudo-pregnant foster mother. The liveborn mice can then be screened for integrants using analysis of tail DNA for the presence of human tumour suppressor gene sequences. The transgene can be either a complete genomic sequence injected as a YAC, BAC, PAC or other chromosome DNA fragment, a cDNA with either the natural promoter or a heterologous promoter, or a minigene containing all of the coding region and other elements found to be necessary for optimum expression.

According to still another aspect of the invention there is provided the use of genetically modified non-human animals as described above for the screening of candidate pharmaceutical compounds.

It will be clearly understood that, although a number of prior art publications are referred to herein, this reference does not constitute an admission that any of these documents forms part of the common general knowledge in the art, in Australia or in any other country. Throughout this specification and the claims, the words “comprise”, “comprises” and “comprising” are used in a non-exclusive sense, except where the context requires otherwise.

### BRIEF DESCRIPTION OF THE DRAWINGS

FIG. 1. Schematic representation of tumours with interstitial and terminal allelic loss on chromosome arm 16q in the two series of tumour samples. Polymorphic markers are listed according to their order on 16q from centromere to telomere and the markers used for each series are indicated by X. Tumour identification numbers are shown at the top of each column. At the right of the figure, the three smallest regions of loss of heterozygosity are indicated.

### MODES FOR PERFORMING THE INVENTION

### EXAMPLE 1

### Collection of Breast Cancer Patient Material

Two series of breast cancer patients were analysed for this study. Histopathological classification of each tumour specimen was carried out by our collaborators according to World Health Organisation criteria (WHO, 1981). Patients were graded histopathologically according to the modified Bloom and Richardson method (Elston and Ellis, 1990) and patient material was obtained upon approval of local Medical Ethics Committees. Tumour tissue DNA and peripheral blood DNA from the same individual was isolated as previously described (Devilee et al., 1991) using standard laboratory protocols.

Series 1 consisted of 189 patients operated on between 1986 and 1993 in three Dutch hospitals, a Dutch University and two peripheral centres. Tumour tissue was snap frozen within a few hours of resection. For DNA isolation, a tissue block was selected only if it contained at least 50% of tumour cells following examination of haematoxilin and eosin stained tissue sections by a pathologist. Tissue blocks that contained fewer than 50% of tumour cells were omitted from further analysis.

Series 2 consisted of 123 patients operated on between 1987 and 1997 at the Flinders Medical Centre in Adelaide, Australia. Of these, 87 were collected as fresh specimens within a few hours of surgical resection, confirmed as malignant tissue by pathological analysis, snap frozen in liquid nitrogen, and stored at −70° C. The remaining 36 tumour tissue samples were obtained from archival paraffin embedded tumour blocks. Prior to DNA isolation, tumour cells were microdissected from tissue sections mounted on glass slides so as to yield at least 80% tumour cells. In some instances, no peripheral blood was available such that pathologically identified paraffin embedded non-malignant lymph node tissue was used instead.

### EXAMPLE 2

### LOH Analysis of Chromosome 16q Markers in Breast cancer samples

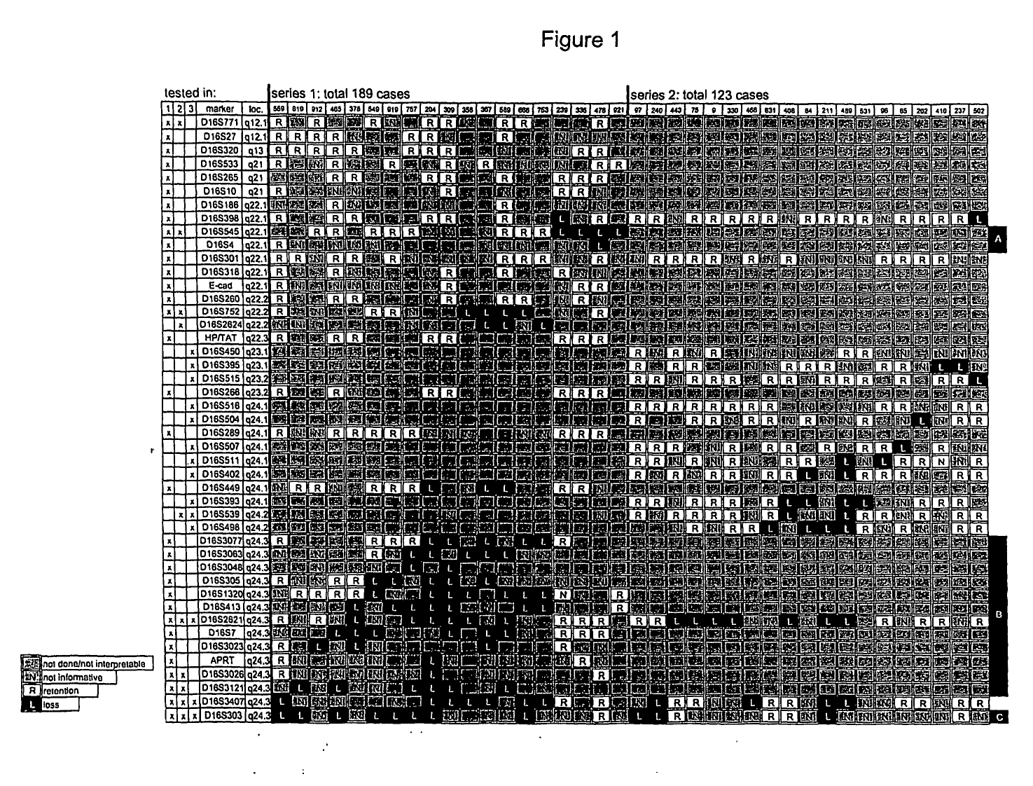

In order to identify the location of tumour suppressor genes associated with breast cancer, LOH analysis of tumour samples was conducted. A total of 45 genetic markers mapping to chromosome 16 were used for the LOH analysis of the breast tumour and matched normal DNA samples collected for this study. FIG. 1 indicates for which tumour series they were used and their cytogenetic location. Details regarding all markers can be obtained from the Genome Database (GDB) at http://www.gdb.org. The physical order of markers with respect to each other was determined from a combination of information in GDB, by mapping on a chromosome 16 somatic cell hybrid map (Callen et al., 1995) and by genomic sequence information.

Four alternative methods were used for the LOH analysis:

1) For RFLP and VNTR markers, Southern blotting was used to test for allelic imbalance. These markers were used on only a subset of samples. Methods used were as previously described (Devilee et al., 1991).

2) Microsatellite markers were amplified from tumour and normal DNA using the polymerase chain reaction (PCR) incorporating standard methodologies (Weber and May, 1989; Sambrook et al., 1989). A typical reaction consisted of 12 ul and contained 100 ng of template, 5 pmol of both primers, 0.2 mM of each dNTP, 1 μcurie [α-³²P]dCTP, 1.5 mM MgCl₂, 1.2 ul Supertag buffer and 0.06 units of Supertag (HT biotechnologies). A Phosphor Imager type 445 SI (Molecular Dynamics, Sunnyvale, Calif.) was used to quantify ambiguous results. In these cases, the Allelic Imbalance Factor (AIF) was determined as the quotient of the peak height ratios from the normal and tumour DNA pair. The threshold for allelic imbalance was defined as a 40% reduction of one allele, agreeing with an AIF of ≧1.7 or ≦0.59. This threshold is in accordance with the selection of tumour tissue blocks containing at least 50% tumour cells with a 10% error-range. The threshold for retention has been previously determined to range from 0.76 to 1.3 (Devilee et al., 1994). This leaves a range of AIFs (0.58-0.75 and 1.31-1.69) for which no definite decision has been made. This “grey area” is indicated by grey boxes in FIG. 1 and tumours with only “grey area” values were discarded completely from the analysis.

3) The third method for determining allelic imbalance was similar to the second method above, however radioactively labelled dCTP was omitted. Instead, PCR of polymorphic microsatellite markers was done with one of the PCR primers labelled fluorescently with FAM, TET or HEX. Analysis of PCR products generated was on an ABI 377 automatic sequencer (PE Biosystems) using 6% polyacrylamide gels containing 8M urea. Peak height values and peak sizes were analysed with the GeneScan programme (PE Biosystems). The same thresholds for allelic imbalance, retention and grey areas were used as for the radioactive analysis.

4) An alternative fluorescent-based system was also used. In this instance PCR primers were labelled with fluorescein or hexachlorofluorescein. PCR reaction volumes were 20 μl and included 100 ng of template, 100 ng of each primer, 0.2 mM of each dNTP, 1-2 mM MgCl₂, 1× AmpliTaq Gold buffer and 0.8 units AmpliTaq Gold enzyme (Perkin Elmer). Cycling conditions were 10 cycles of 94° C. for 30 seconds, 60° C. for 30 seconds, 72° C. for 1 minute, followed by 25 cycles of 94° C. 30 seconds, 55° C. for 30 seconds, 72° C. for 1 minute, with a final extension of 72° C. for 10 minutes. PCR amplimers were analysed on an ABI 373 automated sequencer (PE Biosystems) using the GeneScan programme (PE Biosystems). The threshold range of AIF for allele retention was defined as 0.61-1.69, allelic loss as ≦0.5 or ≧2.0, or the “grey area” as 051-0.6 or 1.7-1.99.

The first three methods were applied to the first tumour series while the last method was adopted for the second series of tumour samples. For statistical analysis, a comparison of allelic imbalance data for validation of the different detection methods and of the different tumour series was done using the Chi-square test.

The identification of the smallest region of overlap (SRO) involved in LOH is instrumental for narrowing down the location of the tumour suppressor gene targeted by LOH. FIG. 1 shows the LOH results for tumour samples, which displayed small regions of loss (ie interstitial and telomeric LOH) and does not include samples that showed complex LOH (alternating loss and retention of markers). When comparing the two sample sets at least three consistent regions emerge with two being at the telomere in band 16q24.3 and one at 16q22.1. The region at 16q22.1 is defined by the markers D16S398 and D16S301 and is based on the interstitial LOH events seen in three tumours from series 1 (239/335/478) and one tumour from series 2 (237). At the telomere (16q24.2-16q24.3), the first region is defined by the markers D16S498 and D16S3407 and is based on four tumours from series 2 (443/75/631/408) while the second region (16q24.3) extends from D16S3407 to the telomere and is based on one tumour from series 1 (559) and three from series 2 (97/240/466). LOH limited to the telomere but involving both of the regions identified at this site could be found in an additional 17 tumour samples.

Other studies have shown that the long arm of chromosome 16 is also a target for LOH in prostate, lung, hepatocellular, ovarian, rhabdomyosarcoma and Wilms' tumours. Detailed analysis of prostate carcinomas has revealed an overlap in the smallest regions of LOH seen in this cancer to that seen with breast cancer which suggests that 16q harbours a tumour suppressor gene implicated in many tumour types.

### EXAMPLE 3

### Construction of a Physical Map of 16q24.3

To identify novel candidate breast cancer tumour suppressor genes mapping to the smallest regions of overlap at 16q24.3, a clone based physical map contig covering this region was needed. At the start of this phase of the project the most commonly used and readily accessible cloned genomic DNA fragments were contained in lambda, cosmid or YAC vectors. During the construction of whole chromosome 16 physical maps, clones from a number of YAC libraries were incorporated into the map (Doggett et al., 1995). These included clones from a flow-sorted chromosome 16-specific YAC library (McCormick et al., 1993), from the CEPH Mark I and MegaYAC libraries and from a half-telomere YAC library (Riethman et al., 1989). Detailed STS and Southern analysis of YAC clones mapping at 16q24.3 established that very few were localised between the CY2/CY3 somatic cell hybrid breakpoint and the long arm telomere. However, those that were located in this region gave inconsistent mapping results and were suspected to be rearranged or deleted. Coupled with the fact that YAC clones make poor sequencing substrates, and the difficulty in isolating the cloned human DNA, a physical map based on cosmid clones was the initial preferred option.

A flow-sorted chromosome 16 specific cosmid library had previously been constructed (Longmire et al., 1993), with individual cosmid clones gridded in high-density arrays onto nylon membranes. These filters collectively contained ˜15,000 clones representing an approximately 5.5 fold coverage of chromosome 16. Individual cosmids mapping to the critical regions at 16q24.3 were identified by the hybridisation of these membranes with markers identified by this and previous studies to map to the region. The strategy to align overlapping cosmid clones was based on their STS content and restriction endonuclease digestion pattern. Those clones extending furthest within each initial contig were then used to walk along the chromosome by the hybridisation of the ends of these cosmids back to the high-density cosmid grids. This process continued until all initial contigs were linked and therefore the region defining the location of the breast cancer tumour suppressor genes would be contained within the map. Individual cosmid clones representing a minimum tiling path in the contig were then used for the identification of transcribed sequences by exon trapping, and for genomic sequencing.

Chromosome 16 was sorted from the mouse/human somatic cell hybrid CY18, which contains this chromosome as the only human DNA, and Sau3A partially digested CY18 DNA was ligated into the BamHI cloning site of the cosmid sCOS-1 vector. All grids were hybridised and washed using methods described in Longmire et al. (1993). Briefly, the 10 filters were pre-hybridised in 2 large bottles for at least 2 hours in 20 ml of a solution containing 6×SSC; 10 mM EDTA (pH 8.0); 10× Denhardt's; 1% SDS and 100 μg/ml denatured fragmented salmon sperm DNA at 65° C. Overnight hybridisations with [α-³²P]dCTP labelled probes were performed in 20 ml of fresh hybridisation solution at 65° C. Filters were washed sequentially in solutions of 2×SSC; 0.1% SDS (rinse at room temperature), 2×SSC; 0.1% SDS (room temperature for 15 minutes), 0.1×SSC; 0.1% SDS (room temperature for 15 minutes), and 0.1×SSC; 0.1% SDS (twice for 30 minutes at 50° C. if needed). Membranes were exposed at −70° C. for between 1 to 7 days.

Initial markers used for cosmid grid screening were those known to be located below the somatic cell hybrid breakpoints CY2/CY3 and the long arm telomere (Callen et al., 1995). These included three genes, CMAR, DPEP1, and MC1R; the microsatellite marker D16S303; an end fragment from the cosmid 317E5, which contains the BBC1 gene; and four cDNA clones, yc81e09, yh09a04, D16S532E, and ScDNA-C113. The IMAGE consortium cDNA clone, yc81e09, was obtained through screening an arrayed normalised infant brain oligo-dT primed cDNA library (Soares et al., 1994), with the insert from cDNA clone ScDNA-ASS. Both the ScDNA-A55 and ScDNA-C113 clones were originally isolated from a hexamer primed heteronuclear cDNA library constructed from the mouse/human somatic cell hybrid CY18 (Whitmore et al., 1994). The IMAGE cDNA clone yh09a04 was identified from direct cDNA selection of the cosmid 37B2 which was previously shown to map between the CY18A(D2) breakpoint and the 16q telomere. The EST, D16S532E, was also mapped to the same region. Subsequent to these initial screenings, restriction fragments representing the ends of cosmids were used to identify additional overlapping clones.

Contig assembly was based on methods previously described (Whitmore et al., 1998). Later during the physical map construction, genomic libraries cloned into BAC or PAC vectors (Genome Systems or Rosewell Park Cancer Institute) became available. These libraries were screened to aid in chromosome walking or when gaps that could not be bridged by using the cosmid filters were encountered. All BAC and PAC filters were hybridised and washed according to manufacturers recommendations. Initially, membranes were individually pre-hybridised in large glass bottles for at least 2 hours in 20 ml of 6×SSC; 0.5% SDS; 5× Denhardt's; 100 μg/ml denatured salmon sperm DNA at 65° C. Overnight hybridisations with [α-³²P]dCTP labelled probes were performed at 65° C. in 20 ml of a solution containing 6×SSC; 0.5% SDS; 100 μg/ml denatured salmon sperm DNA. Filters were washed sequentially in solutions of 2×SSC; 0.5% SDS (room temperature 5 minutes), 2×SSC; 0.1% SDS (room temperature 15 minutes) and 0.1×SSC; 0.5% SDS (37° C. 1 hour if needed). PAC or BAC clones identified were aligned to the existing contig based on their restriction enzyme pattern or formed unique contigs which were extended by additional filter screens.

A high-density physical map consisting of cosmid, BAC and PAC clones was established, which extended approximately 3 Mb from the telomere of the long arm of chromosome 16. This contig extends beyond the CY2/CY3 somatic cell hybrid breakpoint and includes the 2 regions of minimal LOH identified at the 16q24.3 region in breast cancer samples. To date, a single gap of unknown size exists in the contig and will be closed by additional contig extension experiments. The depth of coverage has allowed the identification of a minimal tiling path of clones which were subsequently used as templates for gene identification methods such as exon trapping and genomic DNA sequencing.

### EXAMPLE 4

### Identification of Candidate Tumour Suppressor Genes by Analysis of Genomic DNA Sequence

Selected minimal overlapping BAC and PAC clones from the physical map contig were sequenced in order to aid in the identification of candidate breast cancer genes. DNA was prepared from selected clones using a large-scale DNA isolation kit (Qiagen). Approximately 25-50 ug of DNA was then sheared by nebulisation (10 psi for 45 seconds) and blunt ended using standard methodologies (Sambrook et al., 1989). Samples were then run on an agarose gel in order to isolate DNA in the 2-4 Rb size range. These fragments were cleaned from the agarose using QIAquick columns (Qiagen), ligated into puc18 and used to transform competent DH10B or DH5a E. coli cells. DNA was isolated from transformed clones and was sequenced using vector specific primers on an ABI377 sequencer.

Analysis of genomic sequence was performed using PHRED, PHRAP and GAP4 software on a SUN workstation. To assist in the generation of large contigs of genomic sequence, information present in the high-throughput genomic sequence (htgs) database at NCBI was incorporated into the assembly phase of the sequence analysis. The resultant genomic sequence contigs were masked for repeats and analysed using the BLAST algorithm (Altschul et al., 1997) to identify nucleotide and protein homology to sequences in the GenBank non-redundant and EST databases at NCBI. The genomic sequence was also analysed for predicted gene structure using the GENSCAN program and specific screening of the mouse EST dataset was utilised to identify potential human orthologues that have poor representation in the human EST dataset.

Following the identification of homologous EST sequences, in silico cDNA walking experiments were initiated through further dbEST database screening. This was to identify overlapping cDNA sequences present in dbEST that would allow extension of the originally identified partial gene sequence. Overlapping EST sequences were assembled using the DNAStar LaserGene sequence assembly software. Homologous IMAGE cDNA clones in some instances were also purchased and sequenced. These longer stretches of sequence were then compared to known genes by nucleotide and amino acid sequence comparisons using the above procedures.

From in silico analysis of the dbEST database at NCBI using all genomic sequence obtained for the 16q24.3 critical LOH region, a total of 55 gene fragments or gene “signatures” were identified. In the majority of cases each novel gene fragment was represented by a distinct UniGene cluster composed of one or a number of overlapping cDNA clones. The majority of these UniGene clusters appeared to represent the 3′ untranslated regions of their representative gene as their sequence was continuous with the genomic sequence and further in silico manipulation failed to identify open reading frames representing amino acid coding regions.

As well as the 55 gene signatures that were identified in the 16q24.3 region analysed, a total of 48 partial or full-length genes were also present based on in silico analysis of the genomic DNA generated.

Those sequences that are expressed in the breast were considered to be the most likely candidate breast cancer genes. Those genes whose function could implicate it in the tumourigenic process, as predicted from homology searches with known proteins, were treated with the highest priority. Further evidence that a particular candidate is the responsible gene comes from the identification of defective alleles of the gene in affected individuals or from analysis of the expression levels of a particular candidate gene in breast cancer samples compared with normal control tissues.

Table 1 lists those genes or partial gene fragments that were identified to encode functional domains indicative of a potential role in the tumourigenic process. These genes were treated as tumour suppressor genes mapping to the 16q24.3 LOH interval. These genes are represented by SEQ ID Numbers: 1 to 26 and are described in detail below.

BNO60 represents the PRDM7 gene which encodes a protein containing a SET domain and four C2H2 type zinc finger domains. This domain composition is characteristic to the PRDM protein family, which includes 17 proteins. This family is thought to play an important role in chromatin-mediated gene regulation, in development, and in cancer. The majority of studies have focused on the function of PRDM2. This gene is frequently deleted in human cancers, and its re-introduction in tumours causes growth suppression and apoptosis in animal tumour models. The SET domain has recently been shown to possess methyltransferase activity. This finding suggests that the PRDM proteins may influence gene expression by methylation of chromatin. On the basis of the domain composition of PRDM7, its similarity to the demonstrated tumour suppressor protein PRDM2, and its possible role in gene expression regulation as a chromatin methyltransferase, PRDM7 is an ideal tumour suppressor gene candidate.

A group of six genes were identified to encode proteins containing zinc finger domains. These included BNO224, BNO36, BNO44, BNO34, BNO208 and BNO230. The zinc finger domain composition in the predicted proteins of these genes suggests a possible role in DNA binding and/or protein complex interactions that may influence gene transcription events. Any protein involved in gene transcription regulation is of central interest in the elucidation of gene expression control pathways in breast cancer.

From database analysis, the EST clones representing BNO224 were wrongly incorporated in the EST cluster representing the Fanconi Anemia A gene (FANCA). Our detailed in silico analysis of the genomic sequence in this region has indicated that the BNO224 gene is distinct and its direction of transcription is opposite to that of FANCA. The two genes converge with their last exons overlapping. The BNO224/FANCA genomic locus is conserved in the mouse with these two genes converging and overlapping (Wong et al, 2000). Analyses of the predicted protein product indicates that this gene codes for a protein that includes 5 zinc finger domains two of which appear to be forming a PHD finger domain. The PHD finger is a C4HC3 zinc-finger-like motif found in nuclear proteins thought to be involved in chromatin-mediated transcriptional regulation. The PHD finger motif is reminiscent of, but distinct from, the C3HC4 type RING finger. The function of this domain is not yet known but in analogy with the LIM domain it could be involved in protein-protein interactions and be important for the assembly or activity of multicomponent complexes involved in transcriptional activation or repression. In similarity to the RING finger and the LIM domain, the PHD finger is thought to bind two zinc ions.

BNO36 was identified through extensive in silico analysis coupled with GENSCAN gene prediction methods. The human genomic sequence at 16q24.3 identified a complete mouse gene sequence (Mm.103673) but failed to identify corresponding human EST sequences. The GENSCAN program was used on the human genomic sequence in this region and predicted exons were identified that overlapped with regions exhibiting homology to the mouse sequence. BNO36 therefore represents the human orthologue of this mouse gene. BNO36 codes for a 320 amino acid protein that exhibits homology to the SNAI1 gene on chromosome 20. SNAI1 has been found to exhibit reduced expression in breast cancer tissue suggesting a protective role for this protein in normal breast tissue.

BNO44 consists of 2 isoforms and was isolated using a combination of cDNA walking, sequencing of IMAGE cDNA clones, homology searches with mouse EST sequences and exon trapping (see Whitmore et al., 1998 for the exon trapping procedure used). The short form of the gene is 794 base pairs in length (SEQ ID NO: 23), consists of 6 exons and codes for a protein of 197 amino acids (SEQ ID NO: 24). The longer version of the gene is 3,525 base pairs in length (SEQ ID NO: 25), consists of 13 exons and codes for a protein of 451 amino acids (SEQ ID NO: 26). This version of the gene is the result of the use of additional 3′ exons not used in the short form of BNO44. In silico analysis of the long form of BNO44 identifies two functional motifs. These include a DAG/PE binding domain incorporating amino acids 138-186 and PHD/LAP motif incorporating amino acids 384-431. The DAG/PE binding domain has been found in a family of serine/threonine protein kinases which include oncoproteins. The PHD/LAP motif is a zinc finger motif found in a group of proteins known to have function in chromatin remodeling, suggesting its role in transcription regulation. The short form of the BNO44 protein does not show sequence homology to any functional motifs present in available databases.

BNO55 corresponds to the CDT1 gene which is required at the initiation step of transcription (replication licensing). This gene mediates the loading of pre-replication complexes on origin-of-replication sites and is destabilized from chromatin once pre-replication complexes have formed. Its potential involvement in the initiation of DNA replication may argue that this is not a good tumour suppressor candidate. However, after initiation licensing this protein dissociates from the replication origin preventing any further initiation events. Mutations in this gene may render the protein incapable of dissociating leading to continuous replication initiation.

BNO229 corresponds to the CHMP1 gene which is translated from an alternative reading frame located in the transcript coding for PRSM1. CHMP1 codes for a component of the Polycomb-group (PcG) that is involved in gene silencing through mechanisms involving modification of chromatin structure. Exogenous expression of CHMP1 results in cell arrest in the S-phase of the cell cycle suggesting a tumour suppressor role of the protein.

BNO228 corresponds to the BTG3 associated nuclear protein (BANP) which has been shown to interact with the BTG3 protein (Birot et al., 2000). Currently no function has been assigned to BANP, but the BTG family members are believed to have an antiproliferative function. For example, BTG3 RNA expression is associated with different cell cycle arrest processes and BTG3 in vitro overexpression is antiproliferative (Yoshida et al, 1998). The underlying mechanism by which BTG family members induce growth inhibition remain undefined. It has been recently hypothesised that BTG2 interacts with the classical tumour suppressor Rb, maintaining its hypophosphorylated state and preventing cell division. (Guardavaccaro et al, 2000).

### EXAMPLE 6

### Analysis of Tumours and Cell Lines for Tumour Suppressor Gene Mutations

Any one of the tumour suppressor genes that have been identified in this study can be screened by single strand conformation polymorphism (SSCP) analysis in DNA isolated from tumours which display restricted LOH for the 16q24.3 region. Mutations specifically identified in these genes in cancerous tissue will confirm an involvement of that gene in the cancer. In this instance DNA isolated from series 1 and series 2 tumours can be used. A number of breast cancer cell lines, or cell lines from other cancer types, may also be screened. Likewise, tissues from other cancer types can be screened by SSCP for disease causing mutations. Cell lines can be purchased from ATCC, grown according to manufacturers conditions, and DNA isolated from cultured cells using standard protocols (Wyman and White, 1980; Sambrook et al., 1989).

To perform mutation analysis of the tumour suppressor genes using the SSCP technique, a number of variations can be employed. For example, tumour suppressor gene exons can be amplified by PCR using flanking intronic primers, which are labeled at their 5′ ends with HEX. Typical PCR reactions are performed in 96-well plates in a volume of 10 ul using 30 ng of template DNA. Cycling conditions involve an initial denaturation step at 94° C. for 3 minutes followed by 35 cycles of 94° C. for 30 seconds, 60° C. for 11/2 minutes and 72° C. for 1^{1/2} minutes. A final extension step of 72° C. for 10 minutes follows. Twenty ul of loading dye comprising 50% (v/v) formamide, 12.5 mM EDTA and 0.02% (w/v) bromophenol blue is added to completed reactions which are subsequently run on 4% polyacrylamide gels and analysed on the GelScan 2000 system (Corbett Research, AUS) according to manufacturers specifications.

Those samples that display a bandshift compared with normal controls are considered to have a different nucleotide composition in the amplicon being analysed compared to that of normal controls. The amplicon can be sequenced in this sample and compared to wild-type sequence to determine the nucleotide differences. Any base changes that are present in a tumour sample but not present in the corresponding normal control sample from the same individual or in other normal individuals most likely represents a deleterious mutation. This is further confirmed if the base change also leads to an amino acid change or the generation of a truncated form of the protein.

### EXAMPLE 7

### Analysis of the Tumour Suppressor Genes

The following methods are used to determine the structure and function of any one of the breast cancer genes.

Biological Studies

Mammalian expression vectors containing tumour suppressor gene cDNA can be transfected into breast, prostate or other carcinoma cell lines that have lesions in the gene. Phenotypic reversion in cultures (eg cell morphology, growth of transformants in soft-agar, growth rate) and in animals (eg tumourigenicity in nude mice) is examined. These studies can utilise wild-type or mutant forms of the tumour suppressor genes. Deletion and missense mutants of these genes can be constructed by in vitro mutagenesis.

Molecular Biological Studies

The ability of any one of the tumour suppressor proteins to bind known and unknown proteins can be examined. These proteins may give an insight as to the biological pathways in which the tumour suppressor proteins participate. In turn, proteins within these pathways may provide suitable targets for therapeutic applications such as screening for small molecule interactors, as well as antisense and antibody-based therapies directed at these interactors.

The principle behind the yeast two-hybrid procedure is that many eukaryotic transcriptional activators, including those in yeast, consist of two discrete modular domains. The first is a DNA-binding domain that binds to a specific promoter sequence and the second is an activation domain that directs the RNA polymerase II complex to transcribe the gene downstream of the DNA binding site. Both domains are required for transcriptional activation as neither domain can activate transcription on its own. In the yeast two-hybrid procedure, the gene of interest or parts thereof (BAIT), is cloned in such a way that it is expressed as a fusion to a peptide that has a DNA binding domain. A second gene, or number of genes, such as those from a cDNA library (TARGET), is cloned so that it is expressed as a fusion to an activation domain. Interaction of the protein of interest with its binding partner brings the DNA-binding peptide together with the activation domain and initiates transcription of the reporter genes. The first reporter gene will select for yeast cells that contain interacting proteins (this reporter is usually a nutritional gene required for growth on selective media). The second reporter is used for confirmation and while being expressed in response to interacting proteins it is usually not required for growth.

Structural Studies

Tumour suppressor recombinant proteins can be produced in bacterial, yeast, insect and/or mammalian cells and used in crystallographical and NMR studies. Together with molecular modeling of the proteins, structure-driven drug design can be facilitated.

### EXAMPLE 8

### Generation of Polyclonal Antibodies Against the Tumour Suppressor Proteins

The knowledge of the nucleotide and amino acid sequence of the tumour suppressor genes and associated proteins allows for the production of antibodies, which selectively bind to these proteins or fragments thereof. Following the identification of mutations in these tumour suppressor genes, antibodies can also be made to selectively bind and distinguish mutant from normal protein. Antibodies specific for mutagenised epitopes are especially useful in cell culture assays to screen for malignant cells at different stages of malignant development. These antibodies may also be used to screen malignant cells, which have been treated with pharmaceutical agents to evaluate the therapeutic potential of the agent.

To prepare polyclonal antibodies, short peptides can be designed homologous to any one of the tumour suppressor amino acid sequences. Such peptides are typically 10 to 15 amino acids in length. These peptides should be designed in regions of least homology to the mouse orthologue to avoid cross species interactions in further down-stream experiments such as monoclonal antibody production. Synthetic peptides can then be conjugated to biotin (Sulfo-NHS-LC Biotin) using standard protocols supplied with commercially available kits such as the PIERCE™ kit (PIERCE). Biotinylated peptides are subsequently complexed with avidin in solution and for each peptide complex, 2 rabbits are immunized with 4 doses of antigen (200 ug per dose) in intervals of three weeks between doses. The initial dose is mixed with Freund's Complete adjuvant while subsequent doses are combined with Freund's Immuno-adjuvant. After completion of the immunization, rabbits are test bled and reactivity of sera assayed by dot blot with serial dilutions of the original peptides. If rabbits show significant reactivity compared with pre-immune sera, they are then sacrificed and the blood collected such that immune sera can separated for further experiments.

### EXAMPLE 9

### Generation of Monoclonal Antibodies Specific for the Tumour Suppressor Proteins

Monoclonal antibodies can be prepared for any one of the tumour suppressor proteins in the following manner. Immunogen comprising an intact breast cancer protein or peptide (wild type or mutant) is injected in Freund's adjuvant into mice with each mouse receiving four injections of 10 to 100 ug of immunogen. After the fourth injection blood samples taken from the mice are examined for the presence of antibody to the immunogen. Immune mice are sacrificed, their spleens removed and single cell suspensions are prepared (Harlow and Lane, 1988). The spleen cells serve as a source of lymphocytes, which are then fused with a permanently growing myeloma partner cell (Kohler and Milstein, 1975). Cells are plated at a density of 2×10⁵ cells/well in 96 well plates and individual wells are examined for growth. These wells are then tested for the presence of specific antibodies by ELISA or RIA using wild type or mutant breast cancer target protein. Cells in positive wells are expanded and subcloned to establish and confirm monoclonality. Clones with the desired specificity are expanded and grown as ascites in mice followed by purification using affinity chromatography using Protein A Sepharose, ion-exchange chromatography or variations and combinations of these techniques.

### INDUSTRIAL APPLICABILITY

The DNA sequences of the present invention are useful in the diagnosis of cancer, or a pre-disposition thereto. Methods of treatment of cancer and methods for screening for drugs are also made available.

TABLE 1
Tumour Suppressor Genes Identified at Chromosome 16q24.3

| | Associated | | SEQ ID |
|---|---|---|---|
| Gene | UniGene Cluster | Descripon | Numbers |
| BNO60 | Hs.278234 | PR domain containing 7 protein (PRDM7) | 1 to 4 |
| BNO224 | None | ESTs | 5, 6 |
| BNO55 | Hs. 122908 | CDT1 DNA replication factor | 7, 8 |
| BNO36 | None | ESTs | 9, 10 |
| BNO34 | Hs.105280 | Homo sapiens, clone MGC:14381, mRNA, complete coding sequence | 11, 12 |
| BNO208 | Hs.54925 | Homo sapiens mRNA for KIAA1858 protein; partial coding sequence | 13, 14 |
| BNO230 | Hs.147733 | Homo sapiens cDNA FLJ31875 fis, clone NT2RP7002450 | 15, 16 |
| BNO229 | None | Charged multivesicular body protein 1/chromatin modifying protein 1 (CHMP1) | 17, 18 |
| BNO228 | Hs.194637 | BTG3 associated nuclear protein (BANP) | 19 to 22 |
| BNO44 | Hs.65021 | ESTs | 23 to 26 |

### REFERENCES

References cited herein are listed on the following pages, and are incorporated herein by this reference. 3

Altschulμ, S F. et al. (1997). Nucleic Acids Res. 25: 3389-3402.

Birot, A. et al. (2000). Gene 253: 189-196.

Brenner, A J. and Aldaz C M. (1995). Cancer Res. 55: 2892-2895.

Callen, D F. et al. (1995). Genomics 29: 503-511.

Chen, T. et al. (1996). Cancer Res. 56: 5605-5609.

Cleton-Jansen, A-M. et al. (1995). Br. J. Cancer 72: 1241-1244.

Cole, S P. et al. (1984). Mol. Cell Biol. 62: 109-120.

Cote, R J. et al. (1983). Proc. Natl. Acad. Sci. USA 80: 2026-2030.

Culver, K. (1996). Gene Therapy: A Primer for Physicians. Second Edition. (Mary Ann Liebert).

Devilee, P. et al. (1991). Oncogene 6: 1705-1711.

Devilee, P. et al. (1994). Genes Chrom. Cancer 11: 71-78.

Devilee, P. and Cornelisse, C J. (1994). Biochimica et Biophysica Acta 1198: 113-130.

Doggett, M A. et al. (1995). Nature 377 Suppl: 335-365.

Elston, C W. and Ellis, I O. (1990). Histopathology 16: 109-118.

Fearon, E R. and Vogelstein, B. (1990). Cell 61: 759-767.

Friedman, T. (1991). In Therapy for Genetic Diseases. T Friedman (Ed). Oxford University Press. pp 105-121.

Futreal, P A. et al. (1994). Science 266: 120-122.

Groden, J. et al. (1991). Cell 66: 589-600.

Guardavaccaro, D. et al. (2000). Mol. Cell Biol. 20: 1797-1815.

Hall, J M. et al. (1990). Science 250: 1684-1689.

Harlow, E. and Lane, D. (1988). Antibodies: A Laboratory Manual (Cold Spring Harbor Laboratory, Cold Spring Harbor, N.Y.).

Heller, R A. et al. (1997). Proc. Natl. Acad. Sci. USA 94: 2150-2155.

Huse, W D. et al. (1989). Science 246: 1275-1281.

Kohler, G. and Milstein, C. (1975). Nature 256: 495-497.

Kozbor, D. et al. (1985). J. Immunol. Methods 81:31-42.

Longmire, J L. et al. (1993). GATA 10: 69-76.

McCormick, M K. et al. (1993). Proc. Natl. Acad. Sci. USA 90: 1063-1067.

Miki, Y. et al. (1994). Science 266: 66-71.

Miki, Y. et al. (1996). Nature Genet. 13: 245-247.

Orlandi, R. et al. (1989). Proc. Natl. Acad. Sci. USA 86: 3833-3837.

Radford, D M. et al. (1995). Cancer Res. 55: 3399-3405.

Riethman, H C. et al. (1989). Proc. Natl. Acad. Sci. USA 86: 6240-6244.

Saito, H. et al. (1993). Cancer Res. 53: 3382-3385.

Sambrook, J. et al. (1989). Molecular cloning: a laboratory manual. Second Edition. (Cold Spring Harbour Laboratory Press, New York).

Scharf, D. et al. (1994). Results Probl. Cell Differ. 20: 125-162.

Schena, M. et al. (1996). Proc. Natl. Acad. Sci. USA 93: 10614-10619.

Soares, M B. et al. (1994). Proc. Natl. Acad. Sci. USA 91: 9228-9232.

Weber, J L. and May, P E. (1989). Am. J. Hum. Genet. 44: 388-396.

Whitmore, S A. et al. (1994). Genomics 20: 169-175.

Whitmore, S A. et al. (1998). Genomics 50: 1-8.

WHO. (1981). Histological Typing of Breast Tumours. Second Edition. (Geneva).

Winter, G. et al. (1991). Nature 349: 293-299.

Wong, J C. et al. (2000). Genomics 67: 273-283.

Wooster, R. et al. (1995). Nature 378: 789-791.

Wooster, R. et al. (1994). Science 265: 2088-2090.

Wyman, A. and White, R. (1980). Proc. Natl. Acad. Sci. USA 77: 6754-6758.

Yoshida, Y. et al. (1998). Oncogene 16: 2687-2693.

1. A method for the diagnosis of a predisposition to cancer, in a patient, comprising the steps of:
(3) establishing the level of expression of a gene selected from the group consisting of PR domain containing 7 protein (PRDM7), CDT1 DNA replication factor (CDT1), charged multivesicular body protein 1/chromoatin modifying protein 1 (CHMP1), BTG3 associated nucleoprotein (BANP), BNO224, BNO36, BNO34, BNO208, BNO230 and BNO44; and
(4) comparing expression of the gene to a baseline established from expression in normal tissue controls;
wherein substantial variance from the baseline indicates that the patient is susceptible to cancer.

2. A method for determining whether a human tissue is predisposed to a neo-plastic transformation, comprising determining whether in a cell from the tissue a nucleic acid molecule selected from the group consisting of PRDM7, CDT1, CHMP1, BANP, BNO224, BNO36, BNO34, BNO208, BNO230 and BNO44 is absent, present in a mutant form or down-regulated through epigenetic mechanisms.

3. A method according to claim 2 wherein the human tissue is human breast tissue.

4. A method as claimed in claim 2 comprising determining whether the encoded polypeptide is absent or expressed at reduced levels.

5. A method as claimed in claim 4 comprising contacting the cell with an antibody for binding the peptide under conditions which permit the antibody to bind the peptide if it is present.

6. A mutant form of PRDM7, CDT1, CHMP1, BANP, BNO224, BNO36, BNO34, BNO208, BNO230 and BNO44 in which the tumour suppressor activity of the gene is compromised.

7. A polypeptide encoded by a mutant form of a gene as defined in claim 6.

8. An antibody to a mutant form of a gene as defined in claim 6.

9. An isolated nucleotide molecule selected from the group consisting of nucleotide molecules having the sequence set forth in SEQ ID NO:5; SEQ ID NO:9; SEQ ID NO:23 and SEQ ID NO:25.

10. An isolated polypeptide having the amino acid sequence set forth in any one of the group consisting of SEQ ID NO:6; SEQ ID NO:10; SEQ ID NO:12; SEQ ID NO:14; SEQ ID NO:16; SEQ ID NO:24 and SEQ ID NO:26.

11. A polypeptide encoded by a gene comprising a nucleotide sequence set forth in any one of the group consisting of SEQ ID NO:5; SEQ ID NO:9, SEQ ID NO:11; SEQ ID NO:13; SEQ ID NO:15; SEQ ID NO:23 and SEQ ID NO:25.

12. An antibody to a polypeptide as defined in claim 11.

13. An antibody to a polypeptide having an amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26.

14. Use of a nucleotide molecule having the nucleotide sequence set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:22 and SEQ ID NO:25 in the diagnosis of cancer or in establishing the prognosis of a patient diagnosed with cancer.

15. Use as claimed in claim 14 wherein the cancer is breast cancer.

16. Use of a polypeptide having an amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26 in the diagnosis of cancer or in establishing the prognosis of a patient diagnosed with cancer.

17. Use of a polypeptide as claimed in claim 11 in the diagnosis of cancer or in establishing the prognosis of a patient diagnosed with cancer.

18. Use as claimed in either one of claims 16 or 17 wherein the cancer is breast cancer.

19. Use of an antibody as claimed in claim 12 in the diagnosis of cancer or in establishing the prognosis of a patient diagnosed with cancer.

20. Use of an antibody as claimed in claim 13 in the diagnosis of cancer or in establishing the prognosis of a patient diagnosed with cancer.

21. Use as claimed in either one of claims 19 or 20 wherein the cancer is breast cancer.

22. A microarray comprising oligonucleotides or longer fragments derived from any one or more of the genes PRDM7, CDT1, CHMP1, BANP, BNO224, BNO36, BNO34, BNO208, BNO230 and BNO44.

23. A method of treating or inhibiting breast cancer in a patient in need of such treatment comprising the steps of administering to said patient a vector capable of expressing a gene selected from the group consisting of PRDM7, CDT1, CHMP1, BANP, BNO224, BNO36, BNO34, BNO208, BNO230 and BNO44.

24. A method of treating or inhibiting cancer in a patient in need of such treatment, said method comprising administering to said patient a compound which increases expression or activity of a gene selected from the group consisting of PRDM7, CDT1, CHMP1, BANP, BNO224, BNO36, BNO34, BNO208, BNO230 and BNO44.

25. Use of a nucleotide molecule having the nucleotide sequence set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:22 and SEQ ID NO:25 in the treatment of cancer through gene therapy.

26. Use of a polypeptide having the amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26 in the treatment of cancer.

27. Use of a polypeptide as claimed in claim 11 in the treatment of cancer.

28. Use of an antibody as claimed in claim 12 in the treatment of cancer.

29. Use of an antibody as claimed in claim 13 in the treatment of cancer.

30. Use as claimed in any one of claims 23 to 29 wherein the cancer is breast cancer.

31. A genetically modified non-human animal in which a gene selected from the group consisting of PRDM7, CDT1, CHMP1, BANP, BNO224, BNO36, BNO34, BNO208, BNO230 and BNO44 has been inactivated by knockout deletion.

32. A genetically modified non-human animal as claimed in claim 31 wherein the animal is selected from the group consisting of rats, mice, hamsters, guineas pigs, rabbits, dogs, cats, goats, sheep, pigs and non-human primates such as monkeys and chimpanzees.

33. A method of screening for candidate drugs which restore tumour suppressor activity, comprising the steps of:
(1) contacting a cell in which expression of a gene selected from the group consisting of PRDM7, CDT1, CHMP1, BANP, BNO224, BNO36, BNO34, BNO208, BNO230 and BNO44 is compromised, with a candidate drug; and
(2) assaying for the level of tumour suppressor activity in the cell.

34. The use of a drug identified by the method of claim 33 in the treatment of cancer.

35. Use of a drug as claimed in claim 34 in the manufacture of a medicament for the treatment of cancer.

36. Use as claimed in any one of claims 34 or 35 wherein the cancer is breast cancer.

37. A compound which increases expression or activity of a gene selected from the group consisting of PRDM7, CDT1, CHMP1, BANP, BNO224, BNO36, BNO34, BNO208, BNO230 and BNO44.

38. An anti-cancer drug when identified by the method of claim 25.

39. A cell comprising an expression vector capable of expressing PRDM7, CDT1, CHMP1, BANP, BNO224, BNO36, BNO34, BNO208, BNO230 and BNO44.

40. A cell transformed with a nucleotide molecule having the sequence set forth in SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19, SEQ ID NO:21, SEQ ID NO:22 and SEQ ID NO:25.

41. A pharmaceutical composition comprising any one or more of the genes selected from the group consisting of PRDM7, CDT1, CHMP1, BANP, BNO224, BNO36, BNO34, BNO208, BNO230 and BNO44, active fragments thereof, their expression products and antibodies to their expression products, and an inert carrier.

42. A pharmaceutical composition comprising any one or more of a polypeptide having the amino acid sequence set forth in SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20, SEQ ID NO:22, SEQ ID NO:24 and SEQ ID NO:26, a polypeptide as claimed in claim 22, an agonist of any such polypeptide, an antibody as claimed in claim 12 or 13, an expression vector according to claim 23, a compound according to claim 24, or a candidate drug identified by the method of claim 33, and an inert carrier.


SEQUENCE LISTING (26 sequences)

SEQ ID NO: 1 - DNA, 2635 bp, Homo sapiens
  CDS (142)..(1362)
atcattcccttactcgaatgaattaaagtacaggattggggctaaataatgtgagtgccacgctctttctgaagctcttatatcaaggaacatgcattacaactttcctaatccctgcttccctcacttccagattgtgagatgtgtcagaacttcttcattgacagctgtgctgctcatgggccccctacatttgtaaaggacagtgcagtggacaaggggcatcccaaccgttcagccctcagtctgcccccggggctgagaattgggccatcaggcatccctcaggctgggcttggagtatggaacgaggcatctgatctgccactgggtctgcactttggcccctatgagggccgaattacagaagacgaagaggcagccaacagtggatattcctggctaatcaccaaggggagaaactgctatgagtatgtggatggaaaagataaatcctcggccaactggatgaggtatgtgaactgtgcccgggatgatgaagagcagaacctggtggccttccagtaccacaggcagatcttctatagaacctgccgagtcattaggccaggctgtgaactgctggtctggtatggggatgagtatggccaggaactgggcatcaagtggggcagcaagtggaagaaagagctcatggcagggagagaaccaaagccagagatccatccatgtccctcatgctgtctggccttttcaagtcaaaaatttctcagtcaacatgtggaacgcaatcactcctctcagaacttcccaggaccatctgcaagaaaacttctccaaccagagaatccctgcccaggggatcagaatcaggagcggcaatattctgatccacgctgctgtaatgacaaaaccaaaggtcaagagatcaaagaaaggtccaaactcttgaataaaaggacatggcagagggagatttcaagggccttttctagcccacccaaaggacaaatggggagctctagagtgggagaaagaatgatggaagaagagtccagaacaggccagaaagtgaatccagggaacacaggcaaattatttgtgggggtaggaatctcaagaattgcgaaagtcaaatatggagagtgtgggcaaggtttcagtgataagtcagatgttattacacaccaaaggacacacacaggggggaagccctacgtctgcagagagtgtggggagggctttagccggaagtcagacctcctcagtcaccagaggacacacacaggggagaagccttatgtctgcagagagtgtgagcggggctttagccggaagtcagtcctcctcattcaccagaggacacacaggggagaagccccagtctgcaggaaggatgagtaagtcattagtaataaaaccttatctcaatagccacaagaagacaaacgtgatcaccacacacttgcacaccccagctctgaggtggcttcagcgaaagtctgctaaccccttacattccccgagagtgtaaagagatcggaaataactaattaaacaaatccgccactttcatgactagagttgaggaagaacaggggatagttctgtaagtgttcgggggacgtcaacatgtgtggttgtttcccgcactgatcccctccattttttgtgttttgcctcctgttctaattaattttgtctccatacatatctgaaccccaagtgtgtacctcattcttcccttatcactgaaggaaggaagagtccagaagggccacagagaactcaaacgttcagttcaagtctccacaggaattcaaccccagaaagacataaacttggagtccatctggtttaattattggagaatcgattcccaagtccaggaagagaaatgtagggttttacagagtcgcagcaggaaagagagctccctggtctcctgggaagtgtgacctcttctaatggacccctctcctctgctgccatactcccccttggctccccctgtctcctctcctgatttcctccaatctctgtagccccagaagtgaacgccagacaggaacacgcatgtgtgtatatatgtgttcacgtgtgctatgtgtgttaagcctgcatgcatgggtgtgggggtatgtgccctctgtgtacgtatctgtgtgagtgtgggggtttcaagggtgtattaggaataacgctcaaaatcctaaggaaattgaatactctgagagaagagagacagaccctctcatactgttttatattgttttatactcagaaaaggaaaaagaagcaaaactaaaggcaggtagcctggcgcctaggaaccagacctgaaaccaaggaaccagacccgaaaccaggcctgggccggcctgacctaagcctggtagttaaaattcgacccctgacctagcaactgatgttatctatagattatagaaagacattgtgaaacttcccggtctgttctgttccactctgaccatcggtgcatgcagcccctgtcacctaccccctgcttgctcaatcgatcacgaccctctcacgtggacccccttagagttgtgagcccttaaaagggacaggaattgctcactcggggagctcggctcttgagacagcagtcttgctgatgctcctggccgaataaaccgcttccttcttt

SEQ ID NO: 2 - protein, 407 aa, Homo sapiens
MCQNFFIDSCAAHGPPTFVKDSAVDKGHPNRSALSLPPGLRIGPSGIPQAGLGVWNEASDLPLGLHFGPYEGRITEDEEAANSGYSWLITKGRNCYEYVDGKDKSSANWMRYVNCARDDEEQNLVAFQYHRQIFYRTCRVIRPGCELLVWYGDEYGQELGIKWGSKWKKELMAGREPKPEIHPCPSCCLAFSSQKFLSQHVERNHSSQNFPGPSARKLLQPENPCPGDQNQERQYSDPRCCNDKTKGQEIKERSKLLNKRTWQREISRAFSSPPKGQMGSSRVGERMMEEESRTGQKVNPGNTGKLFVGVGISRIAKVKYGECGQGFSDKSDVITHQRTHTGGKPYVCRECGEGFSRKSDLLSHQRTHTGEKPYVCRECERGFSRKSVLLIHQRTHRGEAPVCRKDE

SEQ ID NO: 3 - DNA, 2440 bp, Homo sapiens
  CDS (142)..(657)
atcattcccttactcgaatgaattaaagtacaggattggggctaaataatgtgagtgccacgctctttctgaagctcttatatcaaggaacatgcattacaactttcctaatccctgcttccctcacttccagattgtgagatgtgtcagaacttcttcattgacagctgtgctgctcatgggccccctacatttgtaaaggacagtgcagtggacaaggggcatcccaaccgttcagccctcagtctgcccccggggctgagaattgggccatcaggcatccctcaggctgggcttggagtatggaacgaggcatctgatctgccactgggtctgcactttggcccctatgagggccgaattacagaagacgaagaggcagccaacagtggatattcctggctaatcaccaaggggagaaactgctatgagtatgtggatggaaaagataaatcctcggccaactggatgagaaccaaagccagagatccatccatgtccctcatgctgtctggccttttcaagtcaaaaatttctcagtcaacatgtggaacgcaatcactcctctcagaacttcccaggaccatctgcaagaaaacttctccaaccagagaatccctgcccaggggatcagaatcaggagcggcaatattctgatccacgctgctgtaatgacaaaaccaaaggtcaagagatcaaagaaaggtccaaactcttgaataaaaggacatggcagagggagatttcaagggccttttctagcccacccaaaggacaaatggggagctctagagtgggagaaagaatgatggaagaagagtccagaacaggccagaaagtgaatccagggaacacaggcaaattatttgtgggggtaggaatctcaagaattgcgaaagtcaaatatggagagtgtgggcaaggtttcagtgataagtcagatgttattacacaccaaaggacacacacaggggggaagccctacgtctgcagagagtgtgggagggctttagccggaagtcagacctcctcagtcaccagaggacacacacaggggagaagccttatgtctgcagagagtgtgagcggggctttagccggaagtcagtcctcctcattcaccagaggacacacaggggagaagccccagtctgcaggaaggatgagtaagtcattagtaataaaaccttatctcaatagccacaagaagacaaacgtgatcaccacacacttgcacaccccagctctgaggtggcttcagcgaaagtctgctaaccccttacattccccgagagtgtaaagagatcggaaataactaattaaacaaatccgccactttcatgactagagttgaggaagaacaggggatagttctgtaagtgttcgggggacgtcaacatgtgtggttgtttcccgcactgatcccctccattttttgtgttttgcctcctgttctaattaattttgtctccatacatatctgaaccccaagtgtgtacctcattcttcccttatcactgaaggaaggaagagtccagaagggccacagagaactcaaacgttcagttcaagtctccacaggaattcaaccccagaaagacataaacttggagtccatctggtttaattattggagaatcgattcccaagtccaggaagagaaatgtagggttttacagagtcgcagcaggaaagagagctccctggtctcctgggaagtgtgacctcttctaatggacccctctcctctgctgccatactcccccttggctccccctgtctcctctcctgatttcctccaatctctgtagccccagaagtgaacgccagacaggaacacgcatgtgtgtatatatgtgttcacgtgtgctatgtgtgttaagcctgcatgcatgggtgtgggggtatgtgccctctgtgtacgtatctgtgtgagtgtgggggtttcaagggtgtattaggaataacgctcaaaatcctaaggaaattgaatactctgagagaagagagacagaccctctcatactgttttatattgttttatactcagaaaaggaaaaagaagcaaaactaaaggcaggtagcctggcgcctaggaaccagacctgaaaccaaggaaccagacccgaaaccaggcctgggccggcctgacctaagcctggtagttaaaattcgacccctgacctagcaactgatgttatctatagattatagaaagacattgtgaaacttcccggtctgttctgttccactctgaccatcggtgcatgcagcccctgtcacctaccccctgcttgctcaatcgatcacgaccctctcacgtggacccccttagagttgtgagcccttaaaagggacaggaattgctcactcggggagctcggctcttgagacagcagtcttgctgatgctcctggccgaataaaccgcttccttcttt

SEQ ID NO: 4 - protein, 171 aa, Homo sapiens
MCQNFFIDSCAAHGPPTFVKDSAVDKGHPNRSALSLPPGLRIGPSGIPQAGLGVWNEASDLPLGLHFGPYEGRITEDEEAANSGYSWLITKGRNCYEYVDGKDKSSANWMRTKARDPSMSLMLSGLFKSKISQSTCGTQSLLSELPRTICKKTSPTRESLPRGSESGAAIF

SEQ ID NO: 5 - DNA, 2205 bp, Homo sapiens
  CDS (679)..(1881)
acgccgggtcctctaggaacctcgggccgggcagcacccgcgggattctgctggcgtcctccgctgccatgaagcgggaccggctgggccgcttcctgtctcctgggtcgtcccgacagtgcggggcctcggacggcggcggcggcgtcagccggactcggggccgcccttcccttagcggtgggccgagggtggacggggcgacggcgcggcgcgcctggggcccggtggggtcctgcggggacgcgggcgaggacggcgcggacgaggcaggagcaggccgggctctcgccatgggtcactgtcgcctctgccacgggaagttttcctcgagaagcctgcgcagcatctccgagagggcgcctggagcgagcatggagaggccatccgcagaggagcgcgtgctcgtacgggacttccagcgcctgcttggtgtggctgtccgccaggaccccaccttgtctccgtttgtctgcaagagctgccacgcccagttctaccagtgccacagccttctcaagtccttcctgcagagggtcaacgcctccccggctggtcgccggaagccttgtgcaaaggtcggtgcccagcccccaacaggggcagaggagggagcgtgtctggtggatctgatcacatccagcccccagtgcctgcacggcttggtggggtgggtgcatggacatgcggccagctgcggggccctgccccaccttcagaggacactgtcctccgagtactgcggcgtcatccaggtcgtgtggggctgcgaccagggccacgactacaccatggataccagctccagctgcaaggccttcttgctggacagtgcgctggcagtcaagtggccatgggacaaagagacggcgccacggctgccccagcaccgagggtggaaccctggggatgcccctcagacctcccagggtagagggacagggaccccagttggggctgagaccaagaccctgcccagcacggatgtggcccagcctccttcggacagcgacgcggtggggcccaggtcgggcttcccacctcagccaagcctgcccctttgcagggccccagggcagttgggtgagaagcagcttccatcttcaacctcggatgatcggagacgtcttgagtgaagatgaaaatgacaagaagcaaaatgcccagtcttcggacgagtcctttgagccttacccagaaaggaaagtctctggtaagaagagtgaaagcaaagaagccaagaagtctgaagaaccaagaattcggaagaagccgggacccaagcccggatggaagaagaagcttcgttgtgagagggaggagcttcccaccatctacaagtgtccttaccagggctgcacggccgtgtaccgaggcgctgacggcatgaagaagcacatcaaggagcaccacgaggaggtccgggagcggccctgcccccaccctggctgcaacaaggttttcatgatcgaccgctacctgcagcgccacgtgaagctcatccacacagaggtgcggaactatatctgtgacgaatgtggacaaaccttcaagcagcggaagcaccttctcgtccaccaaatgcgacattcgggagccaagcctttgcagtgtgaggtctgtgggttccagtgcaggcagcgggcatccctcaagtaccacatgaccaaacacaaggctgagactgagctggactttgcctgtgaccagtgtggccggcggtttgagaaggcccacaacctcaatgtacacatgtccatggtgcacccgctgacacagacccaggacaaggccctgcccctggaggcggaaccaccacctgggccaccgagcccctctgtgaccacagagggccaggcggtgaagcccgaacccacctgaggacggcagtgaggatgagcacctctagcagcctggactccgcagtggctgtgtcagcctcacccttcgtgtgcacccgcatgggagggtcggagggtgctgcccgcccttggtgctggaggcgggcttggtgtccggctcaagtagccttcctctgctctgggaccagtggtttattttcccgcaaacgctgagtgactcggggccggacagttcataaataattgattcctttccccactaaagcagtcgaggagatttgtaatccactttttagtgcaacaagagctccatgttatgcttgtaataaattatttacacggg

SEQ ID NO: 6 - protein, 400 aa, Homo sapiens
MDMRPAAGPCPTFRGHCPPSTAASSRSCGAATRATTTPWIPAPAARPSCWTVRWQSSGHGTKRRRHGCPSTEGGTLGMPLRPPRVEGQGPQLGLRPRPCPARMWPSLLRTATRWGPGRASHLSQACPFAGPQGSWVRSSFHLQPRMIGDVLSEDENDKKQNAQSSDESFEPYPERKVSGKKSESKEAKKSEEPRIRKKPGPKPGWKKKLRCEREELPTIYKCPYQGCTAVYRGADGMKKHIKEHHEEVRERPCPHPGCNKVFMIDRYLQRHVKLIHTEVRNYICDECGQTFKQRKHLLVHQMRHSGAKPLQCEVCGFQCRQRASLKYHMTKHKAETELDFACDQCGRRFEKAHNLNVHMSMVHPLTQTQDKALPLEAEPPPGPPSPSVTTEGQAVKPEPT

SEQ ID NO: 7 - DNA, 1898 bp, Homo sapiens
  CDS (26)..(1666)
acgcgtccgcgcgcactccgccgccatggagcagcgccgcgtcaccgacttcttcgcgcgccgccgccccgggcccccccgcatcgcgccgcccaagctggcctgccgcacccccagccccgccaggcccgcactccgcgccccggcctccgctaccagtggcagccgcaagcgcgcccgcccgcccgccgcccccggacgcgaccaggccaggccaccggcccgcaggagactgcggctgtcggtggacgaggtttccagccccagtacccccgaggccccagacatcccagcctgcccttctccgggccagaagataaagaaatccaccccggcagcaggtcagccgccccacctgacatccgcgcaggaccaggacaccatctctgagcttgcgtcatgcctgcaacgggcccgggagctgggggcaagagtccgggcgctgaaggccagtgcccaggatgctggggagtcctgcaccccagaggccgagggccgccctgaggagccatgtggcgagaaggcgcccgcctaccagcgcttccatgccctggcccagcccggcctgccgggactcgtgctgccctacaagtaccaggtgctggcggagatgttccgcagcatggacaccatcgtgggcatgctccacaaccgctccgagacgcccacctttgccaaggtccagcggggcgtccaggacatgatgcgtaggcgttttgaggagcgcaatgttggccagatcaaaaccgtgtacccggcctcctaccgcttccgccaggagcgcagtgtccccaccttcaaggatggcgccaggaggtcagattaccagctcaccatcgagccactgctggagcaggaggctgacggagcagccccccagctcacggcctcgcgcctcctgcagcgacggcagatcttcagccagaagctggtggagcacgtcaaggagcaccacaaggccttcctggcctccctgagccccgccatggtggtgccggaggaccagctgacccgctggcacccgcgcttcaacgtggatgaagtacccgacatcgagccggccgcgctgccccagccacccgccacggagaagctcaccactgctcaggaggtgctggcccgggcccgcaacctgatttcacccaggatggagaaggccttgagtcaattggccctgcgctctgctgcgcccagcagccccgggtctcccaggccagcactgccggctaccccaccagccaccccgcctgcagcctctcccagtgctctgaagggggtgtcccaggatctgctggagcggatccgagccaaggaggcacagaagcagctggcacagatgacgcggtgcccggagcaggagcagcggctgcagcgcttagaacggctgcctgagctggcccgcgtgctgcggagcgtctttgtgtccgaacgcaagcctgcgctcagcatggaggtggcctgtgccaggatggtgggcagctgttgtactatcatgagccctggggaaatggagaagcacctgctgctcctctccgagctgctgccggactggctcagcctccaccgcatccgcaccgacacctacgtcaagctggacaaggccgcggacctggcccacatcactgcacgcctggcccaccagacacgtgctgaggaggggctgtgagcctgggggccactgtggacagacgtgggcttcagaagctcgctggcctgggcccaccagcattttcttttatgaacatgatacactttggtcttcctttccccagcgcccctgagggccagaggcagatgtgggctgcaggctgcacagcccgagggtctctggctgcgggcggtgggccccttcatggggctcacctggtggattcacattaaaccggtttctgtgggca

SEQ ID NO: 8 - protein, 546 aa, Homo sapiens
MEQRRVTDFFARRRPGPPRIAPPKLACRTPSPARPALRAPASATSGSRKRARPPAAPGRDQARPPARRRLRLSVDEVSSPSTPEAPDIPACPSPGQKIKKSTPAAGQPPHLTSAQDQDTISELASCLQRARELGARVRALKASAQDAGESCTPEAEGRPEEPCGEKAPAYQRFHALAQPGLPGLVLPYKYQVLAEMFRSMDTIVGMLHNRSETPTFAKVQRGVQDMMRRRFEERNVGQIKTVYPASYRFRQERSVPTFKDGARRSDYQLTIEPLLEQEADGAAPQLTASRLLQRRQIFSQKLVEHVKEHHKAFLASLSPAMVVPEDQLTRWHPRFNVDEVPDIEPAALPQPPATEKLTTAQEVLARARNLISPRMEKALSQLALRSAAPSSPGSPRPALPATPPATPPAASPSALKGVSQDLLERIRAKEAQKQLAQMTRCPEQEQRLQRLERLPELARVLRSVFVSERKPALSMEVACARMVGSCCTIMSPGEMEKHLLLLSELLPDWLSLHRIRTDTYVKLDKAADLAHITARLAHQTRAEEGL

SEQ ID NO: 9 - DNA, 1391 bp, Homo sapiens
  CDS (88)..(1050)
cttcaagcgccggcggacgcagcagtccggacccaggcgcgcccctcccgccccagcccaccccggcctgccgcccgggaggggaacatgccgcgctccttcctggtgaaaacgcactccagccacagggtccccaactaccggcggctggagacgcagagagaaatcaatggtgcctgctctgcctgtggggggctggtggtgcccctcctcccccgagacaaggaggccccttctgtgcccggtgaccttccccagccctgggaccgctcctcggccgtcgcctgcatctccctgcccctcctgccacggatcgaggaagctctgggggcctctgggctggacgccttggaagtcagcgaggtcgaccctcgggccagccgggccgccattgtacccctcaaagacagcctgaaccacctcaacctgcccccactgctggtgctgcccacacggtggtccccgaccttgggcccagaccggcacggggctccggaaaaactgcttggggctgagcggatgccccgagccccgggcggctttgagtgcttccactgccacaaaccctaccacacgctggccgggctggccaggcaccggcagctgcactgccacctgcaggtggggcgtgtcttcacctgcaagtactgcgacaaggagtacaccagcctgggtgccctcaagatgcacatccgcactcacacgctgccctgcacctgcaagatctgtggcaaggccttctccaggccctggttactgcagggccatgtccgcacccacacaggggagaagccctatgcctgctcgcactgcagcagggcctttgccgaccgctccaaccttcgggcccatctgcaaacgcactcagacgccaagaagtaccggtgccggcgctgcaccaagaccttctcccgcatgtccctcctggcgcggcatgaggaagtcagagcctcggggaggtggccgccagcatgggccggcactgccgccggatggctggcaaggctgcctagttccattgcagcagaaatgaacagttctgacttatagtgagcaccgccctgtggcccttcctcagtaggcacaactacctctcagccagcccccgccagcctttggtttggggtctgggacgagctgccccatgtcacacgtctatgtgcatgtgcacacacactcaaacatgtacacacacgtgccctccccacctcactagactctccgggagatggggcaggactgggagagcccacgattggtgatttgggtgtgttgggatgaggcggagtgcctgtgggatttgtcccggtcagagcctcagggggctggggtctcagggcactcagcttcccaggcaataacagccgtggggtaataaatggtctctgcac

SEQ ID NO: 10 - protein, 320 aa, Homo sapiens
MPRSFLVKTHSSHRVPNYRRLETQREINGACSACGGLVVPLLPRDKEAPSVPGDLPQPWDRSSAVACISLPLLPRIEEALGASGLDALEVSEVDPRASRAAIVPLKDSLNHLNLPPLLVLPTRWSPTLGPDRHGAPEKLLGAERMPRAPGGFECFHCHKPYHTLAGLARHRQLHCHLQVGRVFTCKYCDKEYTSLGALKMHIRTHTLPCTCKICGKAFSRPWLLQGHVRTHTGEKPYACSHCSRAFADRSNLRAHLQTHSDAKKYRCRRCTKTFSRMSLLARHEEVRASGRWPPAWAGTAAGWLARLPSSIAAEMNSSDL

SEQ ID NO: 11 - DNA, 1854 bp, Homo sapiens
  CDS (75)..(788)
ggcacagggctgtgactagcgggccggcccgggccaggacagcgggcggcgggcggcgcgggcctggccccgggatggctatgttccgcagcctggtggcctcggctcagcagcggcagccgccggccgggccggcgggcggcgacagcggcctggaggcgcagtacacctgccccatctgcctggaggtctatcaccggcccgtggccatcggcagctgcggccacacgttctgcggggagtgtctccagccctgcctgcaggtgccatccccgctgtgcccactgtgccgcctgcccttcgaccccaagaaggtggacaaggccacccacgtggagaagcagctctcatcctacaaagcgccctgtcgaggctgcaacaaaaaggtgaccctggcaaagatgagagtgcacatttcgtcctgcctgaaggtccaggagcagatggccaactgccccaagttcgtccccgtggtgcccacatcacagcctatccccagcaacatccccaacaggtccaccttcgcctgcccgtactgtggtgcccgcaacctggaccagcaggagctggtgaagcactgtgtggaaagccaccgcagcgaccccaaccgcgtggtgtgccccatctgctcggcaatgccctggggggaccccagctacaagagcgccaacttcctgcagcacctgcttcaccgacacaagttctcctacgacacctttgtggactacagtattgacgaggaggccgccttccaggctgctctggccctgtctctctctgagaactgaagggaagcgcagccacccgcctgcgtctggggtcagggatgtccccgctcctgtgtcgcacctggcacctgctcgggagcgcacctcaccggactgagctcacaggaggagcctgcacccgcgcagaaggggagccggggccgagcctccgggcctgaatacgggccagccgccgaggccgccagagcagggccgcctggtcccaccggcgtcgctgggttcttcggtgcttctggccgagcaggcggcctacttgggcagggctggacgctgggacctggagctgccgccgtctcttcaaagccatgataccccctcgtgggaagaagggaccgacgcgcgagtcgcgctccgcagtcgagccgggaggaacccaggctgctgccctgcccagcccgaccctgccccggccccgcttccaccttgcgcatttggtactggcttttgtgatacttaggaaccctggcatcttttctatattatccagtgtgataatcttttcacgttttatagagcaaagacagagcagttactcttcatattgcaatatctgtgtttgactaggaataatagtatttttatggaacatttacaaaattatattttttaagaaaacaatcaaaacaagcattgggggattggggcaaggatggaaggagcagtggggcagctgccagagctcaggcgagccatggggtctgctgtggggtctgccctggccacccactgtgtgtctgggtccttgaggtttgtacgtttctctttgatgaccaggaagaaatcccagcaccccagccacaggctgtggctgctcccagcagaggtggggccggcagagaaggggcctcctccacccagagtcctggccttggcccgctgtcaccttcaaagctgactgtgccccgctgcgggaggggacggcaccccagtggtggcagagcttgggggcctgggcaggggcccgcttggcgggccgggcaacacgtcaacattcttttctgttcttggcattaattattgctgtctttttttaaaaaaaaaaagtttaaataaaatgtctcagagc

SEQ ID NO: 12 - protein, 237 aa, Homo sapiens
MAMFRSLVASAQQRQPPAGPAGGDSGLEAQYTCPICLEVYHRPVAIGSCGHTFCGECLQPCLQVPSPLCPLCRLPFDPKKVDKATHVEKQLSSYKAPCRGCNKKVTLAKMRVHISSCLKVQEQMANCPKFVPVVPTSQPIPSNIPNRSTFACPYCGARNLDQQELVKHCVESHRSDPNRVVCPICSAMPWGDPSYKSANFLQHLLHRHKFSYDTFVDYSIDEEAAFQAALALSLSEN

SEQ ID NO: 13 - DNA, 5360 bp, Homo sapiens
  CDS (1601)..(3919)
cttccagtctctgctgatgtgatttcagatgggcgcggctccagaccatcccctgcaatggccagttacgcagcctctccgagccactgcctctctgtggaaggagggcctgaggctgacggggagcagccgcctcgcttggccactctgggacctggggtgatggagggtgcagcggagactgaccaggaggctctgtgtgcaggggagactggggcccagaagccacctggagatcggatgctgtgtccagggaggatggatggtgcagctctgggggaacagccaactgggcagaagggagcctcggcaagggggttctggggaccaagagagaccaaggcgttgggtgtgtgcaaagagtctgggagcgagcctgcggaggacagcagcagggcccacagccgatcagaggaaggtgtctgggaggagaacacgccccccttgggccccctgggttttcccgagacttccagctctccggcggacagcaccaccagcagctgcctccagggcctcccggacaacccagacacccagggtggagtccaggggcctgaaggccccactcctgatgcctctggctccagtgccaaggatcctccaagcttgtttgatgatgaggtctctttctcccagctcttccctccaggcggtcgcttgactagaaagaggaacccgcgtgtctacgggaagcgctgtgagaagccggtgctcccgctgccaacccagcccagctttgaggagggcggtgaccccacgctgggcccagcccgcctgcccacggacctcagcgactccagctccctctgcctctgccatgaggacccgtgggaggacgaggatcccgcaggtctgcccgagtccttcctcctggatgggttcctcaatagcagggtgcctggcattgacccctgggcccccggcctcagcctgtgggccctggagcccagcagggaagctggtgcagagaagctgccctcccactgccccgaggacgatcggccggaggccattcctgagctgcacatggtcccagcggcttggcgaggcctggagatgccggcccctgccgatgactcctcctcttctctcggagatgtgagccccgagccccccagcctggagagagaacgctgtgacggtgggcttcccgggaacacccacctgctgccgctccgtgccacggactttgaggtgctcagcaccaagtttgagatgcaagacctgtgctttctgggaccctttgaagaccccgtgggtctccccggccccagcttcttagacttcgagggcacggcgagctcacaggggccacagagccgaaggacagaggaggctgcaggggcagggagggcccaaggcagaggccggccggccaagggcaggcgggcctcctacaagtgcaaagtgtgcttccagcgcttccgcagcctgggcgagctggacctgcacaagctggcccacacgcccgcgccgccgcccacctgctacatgtgcgtggagcgcaggtttggctcgcgggagctgctgcgggggcacctgcaggagaggcacgcgcagagcaaggccgggccctgggcgtgcggcatgtgcctgaaggaggtggccgacgtctggatgtacaacgagcacctgcgtgagcacgcggtccgcttcgcccgcaggggggcaggcgcggaggtccttgggggacctgcccggaggcctggagggcagcagcgctgtcgcccaccttctgaacagcatcacggaacccgcgcccaaacaccacaggggcaagcgctccgccggcaaggccgccgggagcccgggagacccgtgggggcaagagggagaagccaagaaagacagcccgggcgagagggcgaaaccccgggcacgcagcacccccagcaacccagacggggccgcgaccccagacagcgcctctgccaccgccctggctgacgccggcagcccgggcccccccaggacgacccccagcccgtcccccgacccctgggccggcggggagcccctcctgcaagccaccccggtgcacgaggcctgcaaggacccctcccgcgactgccaccactgcgggaagcgcttccccaagcccttcaagctgcagcgccacctggcggtgcacagcccgcagcgcgtctacctgtgcccccggtgcccccgggtctaccccgagcacggggagctgctggcacacctgggcggggcgcacgggctgctggagcggccggagctgcagcacacgccgctgtatgcctgcgagctctgcgccacggttatgcgcatcatcaagaagtccttcgcctgcagctcctgcaactacaccttcgccaagaaggagcagttcgaccgccacatgaacaagcacctcaggggggggcggcagcccttcgcgttccgcggcgtgcggaggccgggagcgccgggacagaaggcccgggccctcgagggcacactgcccagcaaacggcgcagggtggccatgcccggcagtgcccctgggcccggcgaggacaggcctcctccccggggaagcagccccatcctgagtgagggctctctcccggccctgctccacctgtgttcggaggtggctcccagcaccaccaagggatggcccgagaccctagagaggcctgtagaccccgtgacccacccgatcagaggttgtgagctgccatccaaccaccaggagtgtcccccgccgtctctgtctcccttcccagctgccttggctgatggcagaggagactgcgcgctggacggagccctggagaggccagagaacgaggcttccccaggcagccccgggcctcttctccagcaagctctccctctgggggcatctctgccgcggccgggagccagaggccaagatgcggagggaaagagggctcctctcgtgttctcagggaaacgcagggccccgggtgcccgtggcaggtgtgcccctgaccatttccaggaagaccacctacttcagaaagagaaggaggtgtcctcaagccacatggtgtctgagggggggccccgaggcgccttccacaagggcagcgccaccaagcctgcgggctgccagagctcatcaaaggacaggtcggcagcatccacccccagcaaagcactcaagttcccagtgcacccaaggaaggcggtggggagcctggcacccggggagctggcccgtggcacagagaatgggatgaagcccgccacccccaaagccaaacccggccccagctcccagggcagtggaagccctcgccccggcaccaagacaggaggtggcagccagccccagccagccagcgggcagctccagagcgagacagccaccaccccagccaagcccagcttccccagccggagccctgcaccagagaggctccccgctcgagcccaagccaagagctgcaccaaggggccaagggaagctggtgagcaggggccccacgggagcctaggtcccaaggagaagggagagagcagtacgaagaggaaaaagggccaggtcccagggccagccaggagtgaaagtgtggggagcttcgggagagccccctcagcccctgacaagcccccccggacccctcggaagcaggcaactcccagccgcgtgctcccgaccaagcccaagcccaacagccagaacaaacccaggccgccaccatcagagcagcggaaggcagagccgggccacacacagaggaaggacagactgggcaaggccttcccccaggggagacccctgctcaggccccccaagaggggcacagctgtccacggtgctgaacctgccgagccgcacacccaccggacggccgaggcccagagtgacctcctcagccagctcttcgggcagagactaactggcttcaaaatccctttaaagaaagatgcttctgagtaatttctagaagcaagagcctgggaccggagctgggcgttcctgtctcggcctgcctccttggccagctccggctccctgagatggtccactctgtggccacttgacttcttgtgcaactgctcaggccttgatgtcagagctgaggtggtgatgctttgaacagggcccaggtgggcagcattccctttcttgctggaaggctgggggtgaaagacggggccactgcagcccttttgagaccacacagctgttttcttggtaccaagtacttgaagagacagcagcccatcccctcagcccacacccctgcgccctgtgggcaccgacaccacagaagccaatgtttggagatttgcacaacctcccgttcccccacatggagaagggaagtaagttgaggcagccgtgggatggtggtaggttccctcttagtcttgctgctgttgctggaattccaaagtgaccttagaaaccacgtgggggaaggcagtgctcactacttagaagggttgcttctgagccgcctggtcccccaagagcacaacaggcctcctccctctgaccacagggtcatgcctcctccctctgaccacagggtcatgcctcctccctctgaccacagggtcatgccagcctccatttgcgctgcgggagaaaagcccatctctagcacaccttgaccccaggaaccgggttcccgtatggaactgggaagaaaccgcccctgtgccagctcccgcgggccctcctcgttccctcccagcctccatggccgccctctagagcctccctgctgtacggagctctgggctccacctatttgcaatgttactctgaagtttctggtgctatttttgtgttgtaatgtgaatacaggcttccttgattttttttttaatgggggtattgggtgggacagacggggtcagggaggccccaccatggcttgtcgagggcacgggcacctgcatggcggcgctctccctgcctcccctgccgggctgcaagcctgaggtctgtgctgccggacggggatgctcagggctggggctgcagagccgctgccctggccagggcaccctcatgcaccgacccaacccaggcctgggacgcacgtgtcctctcacagcgtcgtgcctgtgaaggtgggtcaaagggtgagagggcttccttctcacccttctctccataagtatcttgaagatccatggtttgttttgctctattgtttagtttttacttgggtgcaatgtgtacgtcaaaagtttttattttgatatttgaaagagaccaaatcaggcccagaccgcctctctggaaggtgttgtaggccattcaaaacgcctccggagtgtcgcaaaccaagtgcggaggggccctgaggttgtactgtaaacatcatagtgacttgtcttttcaaatatattcccactattttcgc

SEQ ID NO: 14 - protein, 772 aa, Homo sapiens
RRWPTSGCTTSTCVSTRSASPAGGQARRSLGDLPGGLEGSSAVAHLLNSITEPAPKHHRGKRSAGKAAGSPGDPWGQEGEAKKDSPGERAKPRARSTPSNPDGAATPDSASATALADAGSPGPPRTTPSPSPDPWAGGEPLLQATPVHEACKDPSRDCHHCGKRFPKPFKLQRHLAVHSPQRVYLCPRCPRVYPEHGELLAHLGGAHGLLERPELQHTPLYACELCATVMRIIKKSFACSSCNYTFAKKEQFDRHMNKHLRGGRQPFAFRGVRRPGAPGQKARALEGTLPSKRRRVAMPGSAPGPGEDRPPPRGSSPILSEGSLPALLHLCSEVAPSTTKGWPETLERPVDPVTHPIRGCELPSNHQECPPPSLSPFPAALADGRGDCALDGALERPENEASPGSPGPLLQQALPLGASLPRPGARGQDAEGKRAPLVFSGKRRAPGARGRCAPDHFQEDHLLQKEKEVSSSHMVSEGGPRGAFHKGSATKPAGCQSSSKDRSAASTPSKALKFPVHPRKAVGSLAPGELARGTENGMKPATPKAKPGPSSQGSGSPRPGTKTGGGSQPQPASGQLQSETATTPAKPSFPSRSPAPERLPARAQAKSCTKGPREAGEQGPHGSLGPKEKGESSTKRKKGQVPGPARSESVGSFGRAPSAPDKPPRTPRKQATPSRVLPTKPKPNSQNKPRPPPSEQRKAEPGHTQRKDRLGKAFPQGRPLLRPPKRGTAVHGAEPAEPHTHRTAEAQSDLLSQLFGQRLTGFKIPLKKDASE

SEQ ID NO: 15 - DNA, 2914 bp, Homo sapiens
  CDS (333)..(1652)
acagctgatccggttcttgcggcggtgcgtgctggcgccggagagttccgcgcgtgtcctcgggctgtccgcggggaagctggtccgggagtgggcgcccgcccgcctgcctcgcgccttgcgcccccggcaccgcttccagaagggaaagtcgccgccgctcgcggtagacccgggcagcgcttccattccgcggagctggaaataggaatccagccatccgtgggtcaggaggaatggagactgtaccttccacatagattcacaagctgccctgcagtggccttggcttcaggaaaggagcattcagacgcttccgtcagcctcccaggatggcagcccctgacctggcccacggaggtcatgtttctagggactcagtctgccttcatgaagaacagacacaggcagcagggatggtggctggctggctgataaattgttaccaggacgcggtgacctttgacgacgtggctgtggacttcacccaggaggaatggactttactggacccatctcagagagacctctacagagatgtgatgctggaaaactacgagaacctggcctcagtagaatggcgacttaaaaccaaagggccagcacttcggcaggatagatcttggttcagagcatcaaatgagacacagacggcaagaagccacaatggagggcagctctgtgaccgcacgcagtgtggagaagctttcagtgaacactcaggcctcagcacacacgtgagaactcaaaatacaggagacagttgtgtgtctaatcattatgaaagggacttttttattccatgccagaaaaccttgttcacaattggagagcagttttccgtgttgggtcagtgtggaaaagccttcagctctactccaaatgttgtttcccagcaagcatgcactcgggacagatctcttgactacagcagctgtggggaagtgtttcttaatcagtcataccttcaggcacgtgcgggaagtcacaacggagaagaaacatggaaatggaagccgtgtgggaaagctctaactcactccatgggctgcgccacacctgttgaaatgcatgccgtcaggaatccccacgtatgtagggaatgtgggaaggcctttaggtacactgcctaccttactggtcgcgtgcaagtccaccctggggagaagccctgtgaattggaagaatgtggaaaagcctcccctgtttcttccagcctaactcaacatgtaagaattcatgctgcagagaaaccctgtgaatgtaaagaatgcggaaaagccttcactggactctcaggtctttctaaacacgtccaaacagaccctggacagaagccctatgaatgtaaggactgtgggaaagcctgcggtgggttttatctactgaatgagcatggaaaaactcacacgagggagaagccctttgcatgtgtggtttgcggaaaatattttagaaattcctcatgccttaataatcatgttcgaattcacactggaataaaacccatacatgcagctactgtgggaaggccttcactgtgcgctgtggccttactagacacgtacgaacacacacgggcgagaagccatacacgtgtaaggactgcgggaaagccttctgtacatcctcgggccttactgagcatgtaaggactcacactggagagaaaccatatgaatgtaaagattgtgggaaatccttcactgtttcttcaagcctgactgagcacgcaagaatccataccggagagaaaccctacgaatgtaagcagtgtggcaaagccttcacagggcgctcaggcctcactaaacacatgcggacacacaccggggagaagccctatgaatgtaaggactgtgggaaagcctacaatagggtttatctactgaatgagcatgtgaaaactcacacagaggagaagccctttacatgtacggtatgcaggaaatccttcagaaattcctcgtgcctgaataagcacattcatattcacactggaataaaaccttatgaatgtaaggactgtgggaaaacattcactgtttcttcgagcctaactgagcacatacgaactcacactggagagaaaccttatgaatgtaaagtatgcggaaaggccttcaccacatcctcacaccttatcgtgcacataagaacccacaccggtgagaaaccctacatatgtaaggagtgtgggaaagcctttgcttcctcctcacaccttatcgaacacagaaggactcatacaggagagaaaccttacatatgtaacgagtgtgggaaagccttccgtgcctcctctcacctgcataaacatggaagaattcacactgggcagaaaccctataaatgtaaggaatgtgggaaagcatacaataggttttatctactaaaagaacatttaaaaacttacactgaagagcaggtttttgtatgtaaggactgtggaaaatcttttaagaattcctcatgccttaaccatcacactcaaattcacactgatgagaaacctttctaatgtaaagaatgtggggaagctgtcagctacactcattcacgttgaagacatgaaagacctctcgttctccagatgtccatgacttgaggaatgtggctaggcaatcagcatctcatcacaacccggcaggcaggaactcaccctggagccctatgcagcagacacagagaaagccctcagtgttctctaaggtcttgctgaatatggatggtatccagtagagagaactctattgatgtgtgatatgcagcagcattgttgctgttctgctcagtactttgatgatcccttgtgatctcacaatgaagaaaaaccttaggagggtgagaattgttgggaagtcattttttacattacatctttcctcaccctttagtaaacgtggtgattgac

SEQ ID NO: 16 - protein, 439 aa, Homo sapiens
MAAPDLAHGGHVSRDSVCLHEEQTQAAGMVAGWLINCYQDAVTFDDVAVDFTQEEWTLLDPSQRDLYRDVMLENYENLASVEWRLKTKGPALRQDRSWFRASNETQTARSHNGGQLCDRTQCGEAFSEHSGLSTHVRTQNTGDSCVSNHYERDFFIPCQKTLFTIGEQFSVLGQCGKAFSSTPNVVSQQACTRDRSLDYSSCGEVFLNQSYLQARAGSHNGEETWKWKPCGKALTHSMGCATPVEMHAVRNPHVCRECGKAFRYTAYLTGRVQVHPGEKPCELEECGKASPVSSSLTQHVRIHAAEKPCECKECGKAFTGLSGLSKHVQTDPGQKPYECKDCGKACGGFYLLNEHGKTHTREKPFACVVCGKYFRNSSCLNNHVRIHTGIKPIHAATVGRPSLCAVALLDTYEHTRARSHTRVRTAGKPSVHPRALLSM

SEQ ID NO: 17 - DNA, 2254 bp, Homo sapiens
  CDS (36)..(626)
ctccctctccgagtgctgctcctgtcattgtggccatggacgataccctgttccagttgaagttcacggcgaagcagctggagaagctggccaagaaggcggagaaggactccaaggcggagcaggccaaagtgaagaaggcccttctgcagaaaaatgtagagtgtgcccgtgtgtatgccgagaacgccatccgcaagaagaacgaaggtgtgaactggcttcggatggcgtcccgcgtagacgcagtggcctccaaggtgcagacagctgtgactatgaagggggtgaccaagaatatggcccaggtgaccaaagccctggacaaggccctgagcaccatggacctgcagaaggtctcctcagtgatggacaggttcgagcagcaggtgcagaacctggacgtccatacatcggtgatggaggactccatgagctcggccaccaccctgaccacgccgcaggagcaggtggacagcctcatcatgcagatcgccgaggagaatggcctggaggtgctggaccagctcagccagctgcccgagggcgcctctgccgtgggcgagagctctgtgcgcagccaggaggaccagctgtcacggaggttggccgccttgaggaactagccgtgccccgccggtgtgcaccgcctctgccccgtgatgtgctggaaggctcctgtcctctccccaccgcgtcttgcctttgtgctgaccccgcggggctgcggccggcagccactctgcgtctctcacctgccaggcctgcgtggccttagggttgttcctgttcttttaggttgggcggtgggtctgtgtcctggtgttgagtttctgcaaatttctgggggtgatttctgtgactctgggcccacagcggggaggccaagaggggccctgtggactttcacccagcactgtgggggccttcagactctggggcagcagacatgctgcttcccatcagccagagggggtcagggctgccctgttgccaaacaactccctgaggcctctccgcaccacctcagcgggcaggaggtcccaccatgtggacagacatagcccaaggaggcaccacaggtctatgtgtgctgggggatgtcaggtgccacccaacgctgtcctggtggtatttacaatgacatcctcctcctccatcactccaggggtggtgtctcggccgcccctaccagctggctgagccccctggcctcctgcgctccctcacttccctcagttcccaaagctgcccagtccatggggacagaaccgtcactcagatccacattcaagtgtgcccaccctgcagtcttcatcctcactcagctgctgcctctggaggtgcctttggccacatgtgctgtgctgtttgtctcctcgacagggagcctgtccaccagcaggctgcggtcccagcgggtgcgtctgcagctcctccccttgggcagcctggttctcccggaggacctttccttggggccctgcttcatgacgatgctgcctgtgtcaccctctaccatctgtaaacaactgggtgccttccccgaccacaccccaatgccttcccagcttggaagccaaggcagctgatgaagggagctcaggagagccgtcttcagctgggctggggttggggctgctgtgaggaaaacctgccattgtggccctggagagtcaccagcagctcttgggaaggacttgctgggaggctgagagaggctttgggcacagcctgctgtcttttccatttcctaaagtttacttcattgtcttgaggcttccaggttttgtttttgtttttgccaaagtagaaaaggcaggtggtgggcggctggcagggagtgcgggtccccgcccctcttcagtcctgccctcccctcctcagtcctgcccaccccgtgcagcccatgctgaggctgcagtggtgtcgtgggtgttacgtgcaggaacgtggagaccctgacgtgggctcactgcgtttggttttcttttcagaacttgggagcccccagggaggggctagtgttggtaggtcctagacgtggttccctccagcctccccaaaatcaaccctggtgttgagagaacgtccttctgtccatcgtgggtaacagccttggggagggtgcagagctctgcagagccatgggccaggtggggctgcctcagtcctgtccccttgggcactgaggagaggggcccattcacctttctcctagaatgctgttgtaaataaacaaatggatccctgg

SEQ ID NO: 18 - protein, 196 aa, Homo sapiens
MDDTLFQLKFTAKQLEKLAKKAEKDSKAEQAKVKKALLQKNVECARVYAENAIRKKNEGVNWLRMASRVDAVASKVQTAVTMKGVTKNMAQVTKALDKALSTMDLQKVSSVMDRFEQQVQNLDVHTSVMEDSMSSATTLTTPQEQVDSLIMQIAEENGLEVLDQLSQLPEGASAVGESSVRSQEDQLSRRLAALRN

SEQ ID NO: 19 - DNA, 2136 bp, Homo sapiens
  CDS (153)..(1562)
gcagagcggcggcttctctcgcgaggacggacgccattatcgcatctccccgacaaacaccacgagaattccgcagcccacacggtgaccaaaagccagccccactgtgagttgaactctttcgtgttgaccggccactctccgtgctctggatgatgtcggaacacgacctggccgatgtggttcagattgcagtggaagacctgagccctgaccacccagttgttttggagaatcatgtagtgacagatgaagacgaacctgctttgaaacgccagcgactagaaatcaattgccaggatccatctataaagtcattcctgtattccatcaaccagacaatctgcttgcggttggatagcattgaagccaaattgcaagccctggaggctacttgtaaatccttagaagaaaagctggatctggtcacgaacaagcagcacagccccatccaggtccccatggtggccggctcccctctcggggcaacccagacgtgcaacaaagtgcgatgcgctgtgcctgggcgtcggcagaacaccattgtggtgaaggtgccgggccaagaagacagccaccacgaggacggggagagcggctcggaggccagcgactctgtgtccagctgtgggcaggcgggcagtcagagcatcgggagcaacgtcacgctcatcaccctgaactcggaagaggactaccccaatggcacctggctgggcgacgagaacaaccccgagatgcgggtacgctgcgccatcatcccctccgacatgctgcacatcagcaccaactgccgcacggccgagaagatggcgctaacgctgctggactacctcttccaccgcgaggtgcaggctgtgtccaacctctcggggcagggcaagcacgggaagaagcagctggacccgctcaccatctacggcatccggtgtcaccttttctataaatttggcatcacagaatccgactggtaccgaatcaagcagagcatcgactccaagtgccgcacggcgtggcggcgcaagcagcggggccagagcctggcggtcaagagcttctcgcggagaacgcccaactcgtcctcctactgcccttcagagccgatgatgagcaccccacctcctgccagcgagctcccgcagccacagccgcagccgcaggccctgcactacgcgctggccaacgcacagcaggtgcagatccaccagatcggagaagacggacaggtgcaagtaatcccacagggacacctccacatcgcccaggtgccgcagggggagcaagtccagatcacgcaggacagcgagggcaacctccagatccatcacgtggggcaggacggtcaggtgctgcagggtgcacagctgatcgccgtggcctcctcggaccccgcggcggcgggcgtggatgggtcgccactccagggcagcgacatccaggttcagtacgtgcagctggcgccagtgagtgaccacacggccggggcacagacggccgaagccctgcagcccacgctacagccggagatgcagctcgagcacggggccatccagattcagtgagcggtgcccatggcaccaggagcccctcgccggctccgcctacggcccggcccccacgcgccctgctctcacggcctcggcacaggcagcggctgcacgtgttctgctgaagtgcgtctgaaggccgctgcctccgcggggaacagcatcctatgaactgaaagagcagccgccgccgcccccagccggagacccctttcgtttgagtcctgctgttggtgtcggagcacgaggggaggcacggtgcggagagcgtcgcatatgcgcgggaaatcaagaactatgatatttttctgtttaaacagctttttttaatttgctatggtgtttataacaaaaaagaaaatttgaaaaaaaaaatcccaggggagtagcaggagccctttgctgtgtgctctgtccagtgtcatgagacgggagccctttgctgtgtgctctgtccagtgtcatgaggcaggtgtttgcaaagccagctctcggttccgatggggtattgctgacctacttttctaggggaaatgctcttaaacactgtaattatgcatttctaatgaaataaaatgtatttatgacc

SEQ ID NO: 20 - protein, 469 aa, Homo sapiens
MMSEHDLADVVQIAVEDLSPDHPVVLENHVVTDEDEPALKRQRLEINCQDPSIKSFLYSINQTICLRLDSIEAKLQALEATCKSLEEKLDLVTNKQHSPIQVPMVAGSPLGATQTCNKVRCAVPGRRQNTIVVKVPGQEDSHHEDGESGSEASDSVSSCGQAGSQSIGSNVTLITLNSEEDYPNGTWLGDENNPEMRVRCAIIPSDMLHISTNCRTAEKMALTLLDYLFHREVQAVSNLSGQGKHGKKQLDPLTIYGIRCHLFYKFGITESDWYRIKQSIDSKCRTAWRRKQRGQSLAVKSFSRRTPNSSSYCPSEPMMSTPPPASELPQPQPQPQALHYALANAQQVQIHQIGEDGQVQVIPQGHLHIAQVPQGEQVQITQDSEGNLQIHHVGQDGQVLQGAQLIAVASSDPAAAGVDGSPLQGSDIQVQYVQLAPVSDHTAGAQTAEALQPTLQPEMQLEHGAIQIQ

SEQ ID NO: 21 - DNA, 2127 bp, Homo sapiens
  CDS (153)..(1556)
gcagagcggcggcttctctcgcgaggacggacgccattatcgcatctccccgacaaacaccacgagaattccgcagcccacacggtgaccaaaagccagccccactgtgagttgaactctttcgtgttgaccggccactctccgtgctctggatgatgtcggaacacgacctggccgatgtggttcagattgcagtggaagacctgagccctgaccacccagttgttttggagaatcatgtagtgacagatgaagacgaacctgctttgaaacgccagcgactagaaatcaattgccaggatccatctataaagtcattcctgtattccatcaaccagacaatctgcttgcggttggatagcattgaagccaaattgcaagccctggaggctacttgtaaatccttagaagaaaagctggatctggtcacgaacaagcagcacagccccatccaggtccccatggtggccggctcccctctcggggcaacccagacgtgcaacaaagtgcgatgcgctgtgcctgggcgtcggcagaacaccattgtggtgaaggtgccgggccaagaagacagccaccacgaggacggggagagcggctcggaggccagcgactctgtgtccagctgtgggcaggcgggcagtcagagcatcgggagcaacgtcacgctcatcaccctgaactcggaagaggactaccccaatggcacctggctgggcgacgagaacaaccccgagatgcgggtacgctgcgccatcatcccctccgacatgctgcacatcagcaccaactgccgcacggccgagaagatggcgctaacgctgctggactacctcttccaccgcgaggtgcaggctgtgtccaacctctcggggcagggcaagcacgggaagaagcagctggacccgctcaccatctacggcatccggtgtcaccttttctataaatttggcatcacagaatccgactggtaccgaatcaagcagagcatcgactccaagtgccgcacggcgtggcggcgcaagcagcggggccagagcctggcggtcaagagcttctcgcggagaacgcccaactcgtcctcctactgcccttcagagccgatgatgagcaccccacctcctgccagcgagctcccgcagccacagccgcagccgcaggccctgcactacgcgctggccaacgcacagcaggtgcagatccaccagatcggagaagacggacaggtgcaagtaggacacctccacatcgcccaggtgccgcagggggagcaagtccagatcacgcaggacagcgagggcaacctccagatccatcacgtggggcaggacggtcaggtgctgcagggtgcacagctgatcgccgtggcctcctcggaccccgcggcggcgggcgtggatgggtcgccactccagggcagcgacatccaggttcagtacgtgcagctggcgccagtgagtgaccacacggccggggcacagacggccgaagccctgcagcccacgctacagccggagatgcagctcgagcacggggccatccagattcagtgagcggtgcccatggcaccaggagcccctcgccggctccgcctacggcccggcccccacgcgccctgctctcacggcctcggcacaggcagcggctgcacgtgttctgctgaagtgcgtctgaaggccgctgcctccgcggggaacagcatcctatgaactgaaagagcagccgccgccgcccccagccggagacccctttcgtttgagtcctgctgttggtgtcggagcacgaggggaggcacggtgcggagagcgtcgcatatgcgcgggaaatcaagaactatgatatttttctgtttaaacagctttttttaatttgctatggtgtttataacaaaaaagaaaatttgaaaaaaaaaatcccaggggagtagcaggagccctttgctgtgtgctctgtccagtgtcatgagacgggagccctttgctgtgtgctctgtccagtgtcatgaggcaggtgtttgcaaagccagctctcggttccgatggggtattgctgacctacttttctaggggaaatgctcttaaacactgtaattatgcatttctaatgaaataaaatgtatttatgacc

SEQ ID NO: 22 - protein, 466 aa, Homo sapiens
MMSEHDLADVVQIAVEDLSPDHPVVLENHVVTDEDEPALKRQRLEINCQDPSIKSFLYSINQTICLRLDSIEAKLQALEATCKSLEEKLDLVTNKQHSPIQVPMVAGSPLGATQTCNKVRCAVPGRRQNTIVVKVPGQEDSHHEDGESGSEASDSVSSCGQAGSQSIGSNVTLITLNSEEDYPNGTWLGDENNPEMRVRCAIIPSDMLHISTNCRTAEKMALTLLDYLFHREVQAVSNLSGQGKHGKKQLDPLTIYGIRCHLFYKFGITESDWYRIKQSIDSKCRTAWRRKQRGQSLAVKSFSRRTPNSSSYCPSEPMMSTPPPASELPQPQPQPQALHYALANAQQVQIHQIGEDGQVQVGHLHIAQVPQGEQVQITQDSEGNLQIHHVGQDGQVLQGAQLIAVASSDPAAAGVDGSPLQGSDIQVQYVQLAPVSDHTAGAQTAEALQPTLQPEMQLEHGAIQIQ

SEQ ID NO: 23 - DNA, 794 bp, Homo sapiens
  CDS (71)..(664)
gtagcggggcggccgggcggatccagcgcagccgggagacagatgcgaggcggcggtcaggtgggatgctatggaatatgatgagaagctggcccgtttccggcaggcccacctcaaccccttcaacaagcagtctgggccgagacagcatgagcagggccctggggaggaggtcccggacgtcactcctgaagaggccctgcctgagctgccccctggggagccggaattccgctgccctgaacgcgtgatggatctcggcctgtctgaggaccacttctcccgccctgtgggtctgttcctggcctctgacgtccagcagctgcggcaggcgatcgaggagtgcaagcaggtgattctggagctgcccgagcagtcggagaagcagaaggatgccgtggtgcgactcatccacctccggctgaagctccaggagctgaaggaccccaatgaggatgagccaaacatccgagtgctccttgagcaccgcttttacaaggagaagagcaagagcgtcaagcagacctgtgacaagtgtaacaccatcatctgggggctcattcagacctggtacacctgcacaggtgggccgagacccagacgaggagtgaggaatgagagagaccaaagttcctgcctccgctgggctcacattcagatgtgagctatccagtgtggtggccactagccaaacatggccagtcaaagttaaattaactaaatatgcatttcctcgttgtactgtctacattggacagggctgttctggatgagaaattaaactagtgaattac

SEQ ID NO: 24 - protein, 197 aa, Homo sapiens
MEYDEKLARFRQAHLNPFNKQSGPRQHEQGPGEEVPDVTPEEALPELPPGEPEFRCPERVMDLGLSEDHFSRPVGLFLASDVQQLRQAIEECKQVILELPEQSEKQKDAVVRLIHLRLKLQELKDPNEDEPNIRVLLEHRFYKEKSKSVKQTCDKCNTIIWGLIQTWYTCTGGPRPRRGVRNERDQSSCLRWAHIQM

SEQ ID NO: 25 - DNA, 3525 bp, Homo sapiens
  CDS (71)..(1426)
gtagcggggcggccgggcggatccagcgcagccgggagacagatgcgaggcggcggtcaggtgggatgctatggaatatgatgagaagctggcccgtttccggcaggcccacctcaaccccttcaacaagcagtctgggccgagacagcatgagcagggccctggggaggaggtcccggacgtcactcctgaagaggccctgcctgagctgccccctggggagccggaattccgctgccctgaacgcgtgatggatctcggcctgtctgaggaccacttctcccgccctgtgggtctgttcctggcctctgacgtccagcagctgcggcaggcgatcgaggagtgcaagcaggtgattctggagctgcccgagcagtcggagaagcagaaggatgccgtggtgcgactcatccacctccggctgaagctccaggagctgaaggaccccaatgaggatgagccaaacatccgagtgctccttgagcaccgcttttacaaggagaagagcaagagcgtcaagcagacctgtgacaagtgtaacaccatcatctgggggctcattcagacctggtacacctgcacagggtgttattaccgctgtcacagtaagtgcttgaacctcatctccaagccctgtgtgagctccaaagtcagccaccaagctgaatacgaactgaacatctgccctgagacagggctggacagccaggattaccgctgtgccgagtgccgggcgcccatctctctgcggggtgtgcccagtgaggccaggcagtgcgactacaccggccagtactactgcagccactgccactggaacgacctggctgtgatccctgcacgcgttgtacacaactgggactttgagcctcgaaaggtttctcgctgcagcatgcgctacctggcgctgatggtgtctcggcccgtactcaggctccgggagatcaaccctctgctgttcagctacgtggaggagctggtggagattcgcaagctgcgccaggacatcctgctcatgaagccgtacttcatcacctgcagggaggccatggaggctcgtctgctgctgcagctccaggatcggcagcattttgtggagaacgacgagatgtactctgtccaggacctcctggacgtgcatgccggccgcctgggctgctcgctcaccgagatccacacgctcttcgccaagcacatcaagctggactgcgagcggtgccaggccaagggcttcgtgtgtgagctctgcagagagggcgacgtgctgttcccgttcgacagccacacgtctgtgtgcgccgactgctccgcggtcttccacagggactgctactacgacaactccaccacttgtcccaagtgtgcccggctcagcctgaggaagcagtcgctcttccaggagccaggtcccgatgtggaggcctagcgccgaggaacagtgctgggcaccccgcctggcccgccaggacccaccctgccaacatcaagttgttccttctgctccggagacccctggggtgcggccctggccccctccacccctgctgggccagagcgggtgggcagtgtcaaggcccgctgtctcccaggtgcttgctgggactcggggcggctgcacctggctgtcacctgggtgtgctgctgtgaggggtccttgcgtggcccccatccttcccccaatgcagaactccatgggcagggagctggggggacatctcacctcccccatggcacagagccctccacacccctggaccagggcatccgggccctagaaattccacagctcccgtcctggccaccctggaagctcatcaggccaagacccggacagagcttcagaggagtgttgagtgacacctgaggatgcggctgcacacactcagccaagggccgagtctcacctgcggtggggtttcggctctgcctgggggctccatccctttcagccactcgtggccttggggatttctggttgtccccagctgggactgttcacagttgtcacctgcagacctgcctctccctggcctgaggttcaaaggcctcatcggatggtcagtacagtggggtcacctgttgtttctatacaacagcagggaaggggccatggagcttttccctgctgggtgctcctgctttggcccagcccacctttcctggtgctccaagctaggaggctgtggccccagcctgaggagggtgtcctggcctccaggtgtgcagcaggggctgtgtgctgggggaggttccagttaggcgatgggatcctgcagtggtctggtggcatttcttggaaccagatttacctgaggagctctgtcctgctccctgtggagggctccagatagctcagaaatgaccagccaatggccttttgtttgggggcctgaggtcaagagagctgagagtattcgctcgactgagcacattcaggaagatcagggcaggcgtgtgggaggtccctcactccacgggacagaggcccctggacagcagaggaaacctacagctctgggtgaggggacacttggctttggtgtttgcactttacagatcctgcggtccacgaggggcctcaggagaggacgtgtcaggacgtggcttcccagccttctgccttgggcagtgggggtgctcctgtctgtccttttcccccacacccctggactgtgcttggctgttggtgcacatggttggcacacggtgggcagagggcagagaatgccactgcttggttattggtcccctttgaccaggaaacccaagaggagacacctcagtcagcagaaaggccacctggctcactggctcattccaggagtgggagagacggcagggtctcctctttgtcctccggcatcaggaaggggatggtgtccactccccactgtggtggctttaggcaaggttcttattgtctgctctgcctcggtttccccatctggaaaatgggggcaggggtcctgacctacctcaggtggaacggtgagcagggaacatgtcggagtccttcagagaatgtgatgtgaggttggatcaacagtgtgggttcctgtcctgtttccccttcctctttggggctgaggaggaggttaaaggccaaatgctgtttcccaacaccccaaagtctgcacacgtctcatgaatgcatcacatttctgtcatatggatattagccattccgaaatctgtgtaatcaacttcacattattcaagttacaaatcactgtgtccatagaaaaactgtgctggtatttgctggacaaagggttgggccccttttatttttacctgccacccagcatctcccccacatgccccttctgggtgacacagccggtaaacggaatcaacgtatggttctttctgtgggtctgtggcacagcaggaagagcccggtgccgccagcaccttgtggaagaccacacatgggtggtcccacagcatgggaccaggctggcctgagggatgcccagttgtaacaatgctgctgtcactgtctcattaaatatacatccttta

SEQ ID NO: 26 - protein, 451 aa, Homo sapiens
MEYDEKLARFRQAHLNPFNKQSGPRQHEQGPGEEVPDVTPEEALPELPPGEPEFRCPERVMDLGLSEDHFSRPVGLFLASDVQQLRQAIEECKQVILELPEQSEKQKDAVVRLIHLRLKLQELKDPNEDEPNIRVLLEHRFYKEKSKSVKQTCDKCNTIIWGLIQTWYTCTGCYYRCHSKCLNLISKPCVSSKVSHQAEYELNICPETGLDSQDYRCAECRAPISLRGVPSEARQCDYTGQYYCSHCHWNDLAVIPARVVHNWDFEPRKVSRCSMRYLALMVSRPVLRLREINPLLFSYVEELVEIRKLRQDILLMKPYFITCREAMEARLLLQLQDRQHFVENDEMYSVQDLLDVHAGRLGCSLTEIHTLFAKHIKLDCERCQAKGFVCELCREGDVLFPFDSHTSVCADCSAVFHRDCYYDNSTTCPKCARLSLRKQSLFQEPGPDVEA